(19) United States
(12) Patent Application Publication
(10) Pub. No.: US 2011/0003858 A1
(43) Pub. Date: Jan. 6, 2011

(54) MULTIMERIC HETEROCYCLIC COMPOUNDS USEFUL AS NEUTROPHIL ELASTASE INHIBITORS

(76) Inventors: Lena Bergström, Lund (SE); Hans Lönn, Lund (SE); Michael Lundkvist, Lund (SE); Peter Sjö, Lund (SE)

Correspondence Address: MORGAN LEWIS & BOCKIUS LLP, 1111 PENNSYLVANIA AVENUE NW, WASHINGTON, DC, 20004, US

(21) Appl. No.: 12/439,850

(22) PCT Filed: Sep. 3, 2007
(86) PCT No.: PCT/SE2007/000766

§ 371 Date: Mar. 4, 2009

(30) Foreign Application Priority Data
Sep. 4, 2006 (SE) 0601812-1

(51) Int. Cl. A61K 31/444; C07D 401/12; C07D 401/14; A61P 29/00; A61P 11/00; A61P 11/06; A61P 19/02; A61P 35/00; A61P 9/10

(52) U.S. Cl. 514/333; 546/261; 546/256; 514/335

(57) ABSTRACT

The invention provides compounds of formula (I) and formula (IV) (M)-(L)-(M) (I) [(M)-(L⁴)]ₜ-G (VI) wherein M, L, L⁴, G and t are as defined in the specification and optical isomers, racemates and tautomers thereof, and pharmaceutically acceptable salts thereof; together with processes for their preparation, pharmaceutical compositions containing them and their use in therapy. The compounds are inhibitors of human neutrophil elastase.

### FIELD OF THE INVENTION

The present invention relates to novel compounds, processes for their preparation, pharmaceutical compositions containing them and their use in therapy.

### BACKGROUND OF THE INVENTION

Elastases are possibly the most destructive enzymes in the body, having the ability to degrade virtually all connective tissue components. The uncontrolled proteolytic degradation by elastases has been implicated in a number of pathological conditions. Human neutrophil elastase (hNE), a member of the chymotrypsin superfamily of serine proteases is a 33-KDa enzyme stored in the azurophilic granules of the neutrophils. In neutrophils the concentration of NE exceeded 5 mM and its total cellular amount has been estimated to be up to 3 pg. Upon activation, NE is rapidly released from the granules into the extracellular space with some portion remaining bound to neutrophil plasma membrane (See Kawabat et al. 2002, Eur. J. Pharmacol. 451, 1-10). The main intracellular physiological function of NE is degradation of foreign organic molecules phagocytosed by neutrophils, whereas the main target for extracellular elastase is elastin (Janoff and Scherer, 1968, J. Exp. Med. 128, 1137-1155). NE is unique, as compared to other proteases (for example, proteinase 3) in that it has the ability to degrade almost all extracellular matrix and key plasma proteins (See Kawabat et al., 2002, Eur. J. Pharmacol. 451, 1-10). It degrades a wide range of extracellular matrix proteins such as elastin, Type 3 and type 4 collagens, laminin, fibronectin, cytokines, etc. (Ohbayashi, H., 2002, Expert Opin. Investig. Drugs, 11, 965-980). NE is a major common mediator of many pathological changes seen in chronic lung disease including epithelial damage (Stockley, R. A. 1994, Am. J. Resp. Crit. Care Med. 150, 109-113).

The destructive role of NE was solidified almost 40 years ago when Laurell and Eriksson reported an association of chronic airflow obstruction and emphysema with deficiency of serum α₁-antitrypsin (Laurell and Eriksson, 1963, Scand. J. Clin. Invest. 15, 132-140). Subsequently it was determined that α₁antitrypsin is the most important endogenous inhibitor of human NE. The imbalance between human NE and endogenous antiprotease is believed to cause excess human NE in pulmonary tissues which is considered as a major pathogenic factor in chronic obstructive pulmonary disease (COPD). The excessive human NE shows a prominent destructive profile and actively takes part in destroying the normal pulmonary structures, followed by the irreversible enlargement of the respiratory airspaces, as seen mainly in emphysema. There is an increase in neutrophil recruitment into the lungs which is associated with increased lung elastase burden and emphysema in α₁-proteinase inhibitor-deficient mice (Cavarra et al., 1996, Lab. Invest. 75, 273-280). Individuals with higher levels of the NE-α₁ protease inhibitor complex in bronchoalveolar lavage fluid show significantly accelerated decline in lung functions compared to those with lower levels (Betsuyaku et al. 2000, Respiration, 67, 261-267). Instillation of human NE via the trachea in rats causes lung haemorrhage, neutrophil accumulation during acute phase and emphysematous changes during chronic phase (Karaki et al., 2002, Am. J. Resp. Crit. Care Med., 166, 496-500). Studies have shown that the acute phase of pulmonary emphysema and pulmonary haemorrhage caused by NE in hamsters can be inhibited by pre-treatment is with inhibitors of NE (Fujie et al.,1999, Inflamm. Res. 48, 160-167).

Neutrophil-predominant airway inflammation and mucus obstruction of the airways are major pathologic features of COPD, including cystic fibrosis and chronic bronchitis. NE impairs mucin production, leading to mucus obstruction of the airways. NE is reported to increase the expression of major respiratory mucin gene, MUC5AC (Fischer, B. M & Voynow, 2002, Am. J. Respir. Cell Biol., 26, 447-452). Aerosol administration of NE to guinea pigs produces extensive epithelial damage within 20 minutes of contact (Suzuki et al., 1996, Am. J. Resp. Crit. Care Med., 153, 1405-1411). Furthermore NE reduces the ciliary beat frequency of human respiratory epithelium in vitro (Smallman et al., 1984, Thorax, 39, 663-667) which is consistent with the reduced mucociliary clearance that is seen in COPD patients (Currie et al., 1984, Thorax, 42, 126-130). The instillation of NE into the airways leads to mucus gland hyperplasia in hamsters (Lucey et al., 1985, Am. Resp. Crit. Care Med., 132, 362-366). A role for NE is also implicated in mucus hypersecretion in asthma. In an allergen sensitised guinea pig acute asthma model an inhibitor of NE prevented goblet cell degranulation and mucus hypersecretion (Nadel et al., 1999, Eur. Resp. J., 13, 190-196).

NE has been also shown to play a role in the pathogenesis of pulmonary fibrosis. NE: α₁-protenase inhibitor complex is increased in serum of patients with pulmonary fibrosis, which correlates with the clinical parameters in these patients (Yamanouchi et al., 1998, Eur. Resp. J. 11, 120-125). In a murine model of human pulmonary fibrosis, a NE inhibitor reduced bleomycin-induced pulmonary fibrosis (Taooka et al., 1997, Am. J. Resp. Crit. Care Med., 156, 260-265). Furthermore investigators have shown that NE deficient mice are resistant to bleomycin-induced pulmonary fibrosis (Dunsmore et al., 2001, Chest, 120, 35S-36S). Plasma NE level was found to be elevated in patients who progressed to ARDS implicating the importance of NE in early ARDS disease pathogenesis. (Donnelly et al., 1995, Am. J. Res. Crit. Care Med., 151, 428-1433). The antiproteases and NE complexed with antiprotease are increased in lung cancer area (Marchandise et al., 1989, Eur. Resp. J. 2, 623-629). Recent studies have shown that polymorphism in the promoter region of the NE gene are associated with lung cancer development (Taniguchi et al., 2002, Clin. Cancer Res., 8, 1115-1120.

Acute lung injury caused by endotoxin in experimental animals is associated with elevated levels of NE (Kawabata, et al., 1999, Am. J. Resp. Crit. Care, 161, 2013-2018). Acute lung inflammation caused by intratracheal injection of lipopolysaccharide in mice has been shown to elevate the NE activity in bronchoalveolar lavage fluid which is significantly inhibited by a NE inhibitor (Fujie et al., 1999, Eur. J. Pharmacol., 374, 117-125; Yasui, et al., 1995, Eur. Resp. J., 8, 1293-1299). NE also plays an important role in the neutrophil-induced increase of pulmonary microvascular permeability observed in a model of acute lung injury caused by tumour necrosis factor α (TNFα) and phorbol myristate acetate (PMA) in isolated perfused rabbit lungs (Miyazaki et al., 1998, Am. J. Respir. Crit. Care Med., 157, 89-94).

A role for NE has also been suggested in monocrotoline-induced pulmonary vascular wall thickening and cardiac hypertrophy (Molteni et al., 1989, Biochemical Pharmacol. 38, 2411-2419). Serine elastase inhibitor reverses the monocrotaline-induced pulmonary hypertension and remodelling in rat pulmonary arteries (Cowan et al., 2000, Nature Medicine, 6, 698-702). Recent studies have shown that serine elastase, that is, NE or vascular elastase are important in cigarette smoke-induced muscularisation of small pulmonary arteries in guinea pigs (Wright et al., 2002, Am. J. Respir. Crit. Care Med., 166, 954-960).

NE plays a key role in experimental cerebral ischemic damage (Shimakura et al., 2000, Brain Research, 858, 55-60), ischemia-reperfusion lung injury (Kishima et al., 1998, Ann. Thorac. Surg. 65, 913-918) and myocardial ischemia in rat heart (Tiefenbacher et al., 1997, Eur. J. Physiol., 433, 563-570). Human NE levels in plasma are significantly increased above normal in inflammatory bowel diseases, for example, Crohn's disease and ulcerative colitis (Adeyemi et al., 1985, Gut, 26, 1306-1311). In addition NE has also been assumed to be involved in the pathogenesis of rheumatoid arthritis (Adeyemi et al., 1986, Rheumatol. Int., 6, 57). The development of collagen induced arthritis in mice is suppressed by a NE inhibitor (Kakimoto et al., 1995, Cellular Immunol. 165, 26-32).

Thus, human NE is known as one of the most destructive serine proteases and has been implicated in a variety of inflammatory diseases. The important endogenous inhibitor of human NE is α₁-antitypsin. The imbalance between human NE and antiprotease is believed to give rise to an excess of human NE resulting in uncontrolled tissue destruction. The protease/antiprotease balance may be upset by a decreased availability of α₁-antitrypsin either through inactivation by oxidants such as cigarette smoke, or as a result of genetic inability to produce sufficient serum levels. Human NE has been implicated in the promotion or exacerbation of a number of diseases such as pulmonary emphysema, pulmonary fibrosis, adult respiratory distress syndrome (ARDS), ischemia reperfusion injury, rheumatoid arthritis and pulmonary hypertension.

Neutrophil elastase inhibitors are disclosed in, inter alia, WO 2004/024700, WO 20041024701, GB 2 392 910, WO 2005/082863, WO 2005/082864, WO 2004/043924, WO 2005/021512, WO 2005/021509, WO 2005/026123 and WO 2005/026124.

### DISCLOSURE OF THE INVENTION

In one aspect the present invention provides a compound of formula (I)

(M)-(L)-(M) (I)

wherein:

either M represents a group M¹ of formula (IIA) or (IIB):

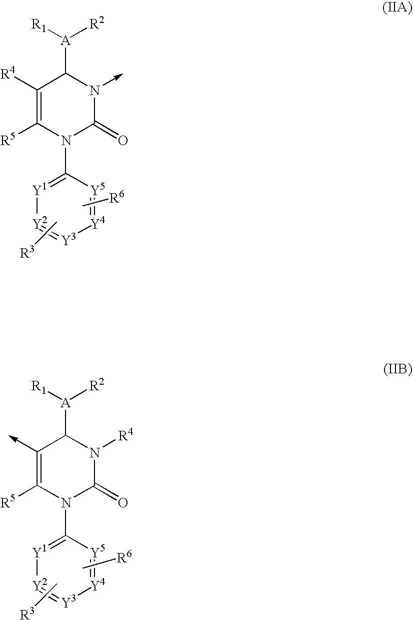

wherein:
A is aryl or heteroaryl;
D is oxygen or sulphur;
R¹, R² and R³ are each independently hydrogen, halogen, nitro, cyano, alkyl, hydroxy or alkoxy; wherein said alkyl and alkoxy may be further substituted with one to three identical or different radicals selected from the group consisting of halogen, hydroxy and alkoxy;
R⁴ is hydrogen, alkyl, trifluoromethylcarbonyl, alkylcarbonyl, alkoxycarbonyl, alkenoxycarbonyl, hydroxycarbonyl, aminocarbonyl, arylcarbonyl, heteroarylcarbonyl, heterocycloalkylcarbonyl, heteroaryl, heterocycloalkyl or cyano; wherein said alkylcarbonyl, alkoxycarbonyl and aminocarbonyl may be further substituted with one to three identical or different radicals selected from the group consisting of cycloalkyl, hydroxy, alkoxy, alkoxycarbonyl, hydroxycarbonyl, aminocarbonyl, cyano, amino, heteroaryl, heterocycloalkyl and tri-(alkyl)-silyl; and wherein said heteroarylcarbonyl, heterocycloalkylcarbonyl, heteroaryl and heterocycloalkyl may be further substituted with alkyl;
or
R⁴ represents a group of Formula (III):

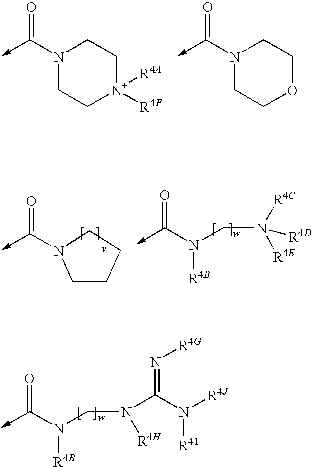

wherein
R^{4A}, R^{4B}, R^{4G}, R^{4H}, R^{4I} and R^{4J} are each independently hydrogen or alkyl; or R^{4H} and R^{4I} may be joined together with the nitrogen atoms to which they are attached to form a ring;
R^{4F} is a lone pair or R^{4F} is alkyl and the nitrogen atom to which it is attached is quaternary and carries a positive charge;
R^{4C}, R^{4D} and R^{4E} are alkyl, or any two of R^{4C}, R^{4D} and R^{4E} may be joined together with the nitrogen atom to which they are attached to form a ring, optionally containing a further heteroatom selected from oxygen or nitrogen;
v is an integer 1 to 3;
w is an integer 1 to 6;
R⁵ is alkyl, which may be optionally substituted with one to three identical or different radicals selected from the group consisting of halogen, hydroxy, alkoxy, alkenoxy, alkylthio, amino, hydroxycarbonyl, alkoxycarbonyl and the radical —O-(alkyl)-O-(alkyl);
or R amino;
R⁶ is halogen, nitro, cyano, alkyl, hydroxy or alkoxy; wherein said alkyl and alkoxy may be further substituted with one to three identical or different radicals selected from the group consisting of halogen, hydroxy and alkoxy;
Y¹, Y², Y³, Y⁴ and Y⁵ are each independently C or N, with the proviso that the ring in which they are comprised contains no more than two N atoms; and
→ indicates the preferred point of attachment of M¹ to the group L;

or M represents a group M² of formula (IV):

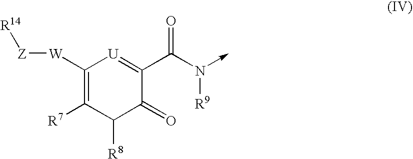

wherein
R⁷ represents hydrogen or alkyl;
U represents N or CR¹⁰;
Either W represents S(O)ₘ wherein m represents an integer 0, 1 or 2; and
Z represents a single bond, —CH₂— or —NR³⁷—; and
R¹⁴ represents a hydrogen atom or OH or a group selected from alkyl and a saturated or unsaturated 3- to 10-membered ring system optionally comprising at least one ring heteroatom selected from nitrogen, oxygen and sulphur; each group being optionally substituted with at least one substituent selected from phenyl, alkoxycarbonyl, halogen, alkyl, alkoxy, CN, OH, NO₂, alkyl substituted by one or more F atoms, alkoxy substituted by one or more F atoms, NR¹²R¹³, C≡CR³⁰, CONR³¹R³², CHO, alkylcarbonyl, S(O)ₚR³³ and OSO₂R³⁴;
Or W represents a 5-membered heterocyclic ring comprising at least one ring heteroatom selected from nitrogen, oxygen and sulphur, wherein at least one of the ring carbon atoms may be optionally replaced by a carbonyl group; and wherein the heterocyclic ring is optionally substituted by at least one substituent selected from halogen, C₁-C₄ alkyl, C₁-C₄ alkoxy, CN, OH, NO₂, C₁-C₃ alkyl substituted by one or more F atoms, C₁-C₃ alkoxy substituted by one or more F atoms, NR⁴⁰R⁴¹, C≡CR⁴⁵, CONR⁴⁶R⁴⁷, CHO, C₂-C₄ alkanoyl, S(O)ₛR⁴⁸ and OSO₂R⁴⁹; and
Z represents a single bond; and
R¹⁴ represents phenyl or a 6-membered heteroaromatic ring comprising 1 to 3 ring nitrogen atoms; said ring being optionally substituted with at least one substituent selected from halogen, C₁-C₄ alkyl, C₁-C₄ alkoxy, CN, OH, NO₂, C₁-C₃ alkyl substituted by one or more F atoms, C₁-C₃ alkoxy substituted by one or more F atoms, NR¹²R¹³, C≡CR³⁰, CONR³¹R³², CHO, C₂-C₄ alkanoyl, S(O)ₚR³³ and OSO₂R³⁴;
R¹², R¹³, R⁴⁰ and R⁴¹ independently represent H, alkyl, formyl or alkylcarbonyl; or the group —NR¹²R¹³ or —NR⁴⁰R⁴¹ together represents a 5 to 7 membered azacyclic ring optionally incorporating one further heteroatom selected from O, S and NR³⁸;
R³⁰ and R⁴⁵ independently represent H, alkyl, Si(CH₃)₃ or phenyl;
R³³ and R³⁴ independently represent H or alkyl; said alkyl being optionally substituted by one or more F atoms;
R¹⁰ represents H or F;
R⁸ represents phenyl or a five- or six-membered heteroaromatic ring containing 1 to 3 heteroatoms independently selected from O, S and N; said ring being optionally substituted with at least one substituent selected from halogen, alkyl, cyano, alkoxy, nitro, methylcarbonyl, NR³⁵R³⁶, alkyl substituted by one or more F atoms or alkoxy substituted by one or more F atoms;
R³⁵, R³⁶, R⁴⁸ and R⁴⁹ independently represent H or alkyl; said alkyl being optionally further substituted by one or more F atoms;
R⁹ represents hydrogen or alkyl optionally substituted with at least one substituent selected from fluoro, hydroxyl and alkoxy;
p is 0, 1 or 2;
s is 0, 1 or 2;
R³¹, R³², R³⁷, R³⁸, R⁴⁶ and R⁴⁷ each independently represent hydrogen or alkyl; and
→ indicates the preferred point of attachment of M² to the group L;

and each group M in formula (I) is selected independently from a group M¹ or M² provided that every compound of formula (I) contains at least one group M²;

L represents a linker group of formula (V):

-L¹-R¹⁵-L²-R¹⁶-L³-X-L³-R¹⁶L²-R¹⁵-L¹- (V)
wherein:
each L¹, each L² and each L³ is independently selected from a direct bond, C(═O), O, NR¹⁷, CONR¹⁸ and NR¹⁹CO;
each R¹⁵ and each R¹⁶ is independently selected from C1 to 10 alkylene or C3 to 7 cycloalkylene; and
X is a direct bond, C(═O), NR²⁰R^{21,} alkylene, cycloalkylene, aryl, aryl¹-aryl², aryl¹-O-aryl², heteroaryl, heteroaryl¹-heteroaryl², heteroaryl¹-O-heteroaryl² i or is selected from the following divalent radicals:

—(O—R²²)ₙ—O —

—N(R²³)—R²²—N(R²⁴)(R²⁵)—R²²—N(R²³)—

—N(R²³)—R²²—N(R²⁷)—C(═NR²⁶)—N(R²⁸)—R²²—N(R²³)—

—N(R²³)—R²²—N(R²³)—

—N(R²⁷)—C(═NR²⁶)—N(R²⁸)—

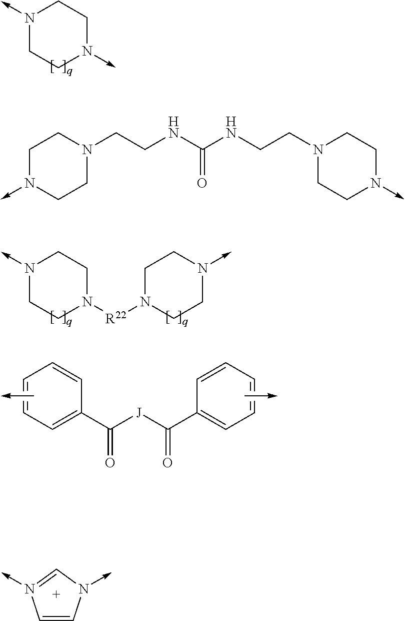

wherein
n is an integer 1 to 4;
each q independently represents an integer 1 or 2;
each R¹⁷, each R¹⁸ and each R¹⁹ are independently selected from H or alkyl;
R²⁰ _{and} R²¹ are independently selected from H and alkyl; and when both represent alkyl, the N atom to which they are attached bears a positive charge; or
R²⁰ and R²¹ are joined together such that the group NR²⁰R²¹ together represents a quaternary 5- to 7-membered azacyclic ring which optionally incorporates one further heteroatom selected from O, N and S;
aryl¹ and aryl² represent the same or different aryl ring systems;
heteroaryl¹ and heteroaryl² represent the same or different heteroaryl ring systems;
each R²² is independently selected from C1 to 10 alkylene or C3 to 7 cycloalkylene;
each R²³, each R²⁶, each R²⁷ and each R²⁸ is independently selected from H or alkyl;
R²⁴ _{and} R²⁵ are independently selected from H and alkyl; and when both represent alkyl, the N atom to which they are attached bears a positive charge; or
R²⁴ _{and} R²⁵ are joined together such that the group NR²⁴R²⁵ together represents a quaternary 5- to 7-membered azacyclic ring which optionally incorporates one further heteroatom selected from O, N and S;
J is selected from the groups —N(R²³)—R²²—N(R²⁴)(R²⁵)—R²²—N(R²³)— or —N(R²³)—R²²—N(R²⁷)—C(═NR²⁶)—(NR²⁸)—R²²—N(R²³)—;

or a pharmaceutically acceptable salt thereof.

In the context of the present specification, unless otherwise stated, an alkyl group or an alkyl moiety in a substituent group (for example, alkoxy) may be linear or branched. Similarly, an alkylene group may be linear or branched. Unless otherwise defined, a ring system may have alicyclic or aromatic properties. An unsaturated ring system may be partially or fully unsaturated.

“Alkylcarbonyl”, “acyl” or “alkanoyl” means a —CO-alkyl group in which the alkyl group is as described herein. Exemplary acyl groups include —COCH₃ and —COCH(CH₃)₂.

“Acylamino” means a —NR-acyl group in which R is H or alkyl and acyl is as described herein. Exemplary acylamino groups include —NHCOCH₃ and —N(CH₃)COCH₃.

is “Alkenoxy” means an —O-alkenyl group in which alkenyl is as described below. Exemplary groups includes —O-allyl (—OCH₂CH═CH₂).

“Alkenoxycarbonyl” means a —COO-alkenyl group which alkenyl is as described below. Exemplary groups includes —C(O)O-allyl.

“Alkoxy” and “alkyloxy” means an —O-alkyl group in which alkyl is as described below. Exemplary alkoxy groups include methoxy (—OCH₃) and ethoxy (OC₂H₅).

“Alkoxycarbonyl” means a —COO-alkyl group in which alkyl is as defined below. Exemplary alkoxycarbonyl groups include methoxycarbonyl and ethoxycarbonyl.

“Alkyl” or “lower alkyl”, as a group or part of a group, refers to a straight or branched chain saturated hydrocarbon group having from 1 to 12, preferably 1 to 6, carbon atoms in the chain. Exemplary alkyl groups include methyl, ethyl, 1-propyl and 2-propyl.

“Alkenyl” as a group or part of a group refers to a straight or branched chain hydrocarbon group having from 1 to 12, preferably 1 to 6, carbon atoms and one carbon-carbon double bond in the chain. Exemplary alkenyl groups include ethenyl, 1-propenyl and 2-propenyl.

“Alkylamino” means a —NH-alkyl group in which alkyl is as defined above. Exemplary alkylamino groups include methylamino and ethylamino.

“Alkylene means an -alkyl-group in which alkyl is as defined previously. Exemplary alkylene groups include —CH₂—, —(CH₂)₂— and —CH(CH₃)CH₂—.

“Alkenylene” means an-alkenyl-group in which alkenyl is as defined previously. Exemplary alkenylene groups include —CH═CH—, —CH═CHCH₂— and —CH₂CH═CH—.

“Alkylthio” means a —S-alkyl group in which alkyl is as defined above. Exemplary alkylthio groups include methylthio and ethylthio.

“Amino” means a —NR¹R² group where R¹ and R² may be independently a hydrogen atom, alkyl, aryl, arylalkyl, alkenyl, alkynyl, heteroaryl or heterocycloalkyl group. That is, the amino group may be primary, secondary or tertiary. Exemplary amino groups include —NH₂, NHCH₃, —NHPh, —N(CH₃)₂, etc.

“Aminocarbonyl” means a —CO—NRR group in which R is as herein described. Exemplary aminocarbonyl groups include —CONH₂, —CONHCH₃ and —CONH-phenyl.

“Aminoalkyl” means an alkyl-NH₂ group in which alkyl is as previously described. Exemplary aminoalkyl groups include —CH₂NH₂.

“Ammonium” means a quaternary nitrogen group —N⁺R¹R²R³ where R¹, R² and R³ are alkyl, aryl, alkenyl, arylalkyl, heteroaryl, heterocycloalkyl, and the nitrogen atom carries a formal positive charge.

“Aryl” as a group or part of a group denotes an optionally substituted monocyclic or multicyclic aromatic carbocyclic moiety of from 6 to 14 carbon atoms, preferably from 6 to 10 carbon atoms, such as phenyl or naphthyl. The aryl group may be substituted by one or more substituent groups.

“Arylalkyl” means an aryl-alkyl-group in which the aryl and alkyl moieties are as previously described. Exemplary arylalkyl groups include benzyl, phenethyl and naphthylmethyl.

“Arylalkyloxy” means an aryl-alkyloxy-group in which the aryl and alkyloxy moieties are as previously described. Preferred arylalkyloxy groups contain a C1-4 alkyl moiety. Exemplary arylalkyl groups include benzyloxy.

“Arylcarbonyl” means an aromatic ring joined to a carbonyl group —(C═O). Exemplary groups include benzoyl (—C(O)Ph).

“Aryloxy” means an —O-aryl group in which aryl is described above. Exemplary aryloxy groups include phenoxy.

“Cyclic amine” means an optionally substituted 3 to 8 membered monocyclic cycloalkyl ring system where one of the ring carbon atoms is replaced by nitrogen, and which may optionally contain an additional heteroatom selected from 0, S or NR (wherein R is as described herein). Exemplary cyclic amines include pyrrolidine, piperidine, morpholine, piperazine and N-methylpiperazine. The cyclic amine group may be substituted by one or more substituent groups.

“Cycloalkyl” means an optionally substituted saturated monocyclic or bicyclic ring system of from 3 to 12 carbon atoms, preferably from 3 to 8 carbon atoms, and more preferably from 3 to 6 carbon atoms. Exemplary monocyclic cycloalkyl rings include cyclopropyl, cyclopentyl, cyclohexyl and cycloheptyl. The cycloalkyl group may be substituted by one or more substituent groups.

“Cyloalkylene” means an optionally substituted saturated monocyclic or bicyclic ring system of from 3 to 12 carbon atoms, preferably from 3 to 8 carbon atoms, and more preferably from 3 to 6 carbon atoms, as a bivalent radical. Exemplary cycloalkylene groups include cyclohexane-1,4-diyl.

“Cycloalkylalkyl” means a cycloalkyl-alkyl-group in which the cycloalkyl and alkyl moieties are as previously described. Exemplary monocyclic cycloalkylalkyl groups include cyclopropylmethyl, cyclopentylmethyl, cyclohexylmethyl and cycloheptylmethyl.

“Dendrimer” means a multifunctional core group with a branching group attached to each functional site. Each branching site can be attached to another branching molecule and this process may be repeated multiple times.

“Halo” or “halogen” means fluoro, chloro, bromo, or iodo.

“Haloalkoxy' means an —O-alkyl group in which the alkyl is substituted by one or more halogen atoms. Exemplary haloalkyl groups include trifluoromethoxy and difluoromethoxy.

“Haloalkyl” means an alkyl group which is substituted by one or more halo atoms. Exemplary haloalkyl groups include trifluoromethyl.

“Heteroaryl” as a group or part of a group denotes an optionally substituted aromatic monocyclic or multicyclic organic moiety of from 5 to 14 ring atoms, preferably from 5 to 10 ring atoms, in which one or more of the ring atoms is/are element(s) other than carbon, for example, nitrogen, oxygen or sulfur. Examples of such groups include benzimidazolyl, benzoxazolyl, benzothiazolyl, benzofuranyl, benzothienyl, furyl, imidazolyl, indolyl, indolizinyl, isoxazolyl, isoquinolinyl, isothiazolyl, oxazolyl, oxadiazolyl, pyrazinyl, pyridazinyl, pyrazolyl, pyridyl, pyrimidinyl, pyrrolyl, quinazolinyl, quinolinyl, tetrazolyl, 1,3,4-thiadiazolyl, thiazolyl, thienyl and triazolyl groups. The heteroaryl group may be substituted by one or more substituent groups. The heteroaryl group may be attached to the remainder of the compound of the invention by any available carbon or nitrogen atom.

“Heteroarylcarbonyl” means a heteroaryl group attached to a carbonyl group, —C(O)—. Exemplary groups are pyridine-2-carbonyl and thiophene-2-carbonyl.

“Heteroaryloxy” means a heteroaryloxy-group in which the heteroaryl is as previously described. Exemplary heteroaryloxy groups include pyridyloxy.

“Heterocycloalkyl” means: (i) an optionally substituted cycloalkyl group of from 4 to 8 ring members which contains one or more heteroatoms selected from 0, S or NR; (ii) a cycloalkyl group of from 4 to 8 ring members which contains CONR or CONRCO (examples of such groups include succinimidyl and 2-oxopyrrolidinyl). The heterocycloalkyl group may be substituted by one or more substituent groups. The heterocycloalkyl group may be attached to the remainder of the compound by any available carbon or nitrogen atom.

“Heterocycloalkylalkyl” means a heterocycloalkyl-alkyl-group in which the heterocycloalkyl and alkyl moieties are as previously described.

“Hydroxycarbonyl” means a group —COOH.

Examples of a 5 to 7 membered azacyclic ring optionally incorporating one further heteroatom selected from O, S and NR³⁸ include pyrrolidine, piperidine, piperazine, morpholine and perhydroazepine.

Examples of 5-membered heterocyclic ring systems that may be used, which may be saturated or partially unsaturated or fully unsaturated include any one of pyrrolidinyl, tetrahydrofuranyl, pyrroline, imidazolidinyl, imidazolinyl, pyrazolidinyl, pyrazolinyl, pyrrolidinonyl, imidazolidinonyl, oxazolyl, pyrazolyl, thiazolidinyl, thienyl, isoxazolyl, isothiazolyl, thiadiazolyl, pyrrolyl, furanyl, thiazolyl, imidazolyl, furazanyl, triazolyl and tetrazolyl.

In one embodiment, each group M in formula (I) represents a group M² and the two M² groups are chosen independently. In another embodiment, each group M in formula (I) represents a group M² and the two M² groups are the same.

In one embodiment, one group M in formula (I) represents a group M¹ and the other group M represents a group M².

In one embodiment, A in formula (IIA) or (IIB) is a phenyl ring.

In one embodiment D in formula (IIA) or (IIB) is an oxygen atom.

In another embodiment, the groups M¹ have the stereochemistry shown below:

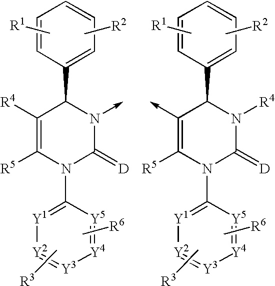

In one embodiment, in formula (IV), W represents S(O)ₘ wherein m represents an integer 0, 1 or 2; and Z represents a single bond, —CH₂— or —NR³⁷—; and R¹⁴ represents a hydrogen atom or OH or a group selected from alkyl and a saturated or unsaturated 3- to 10-membered ring system optionally comprising at least one ring heteroatom selected from nitrogen, oxygen and sulphur; each group being optionally substituted with at least one substituent selected from phenyl, alkoxycarbonyl, halogen, alkyl, alkoxy, CN, OH, NO₂, alkyl substituted by one or more F atoms, alkoxy substituted by one or more F atoms, NR¹²R¹³, C≡CR³⁰, CONR³¹R³², CHO, alkylcarbonyl, S(O)ₚR³³ and OSO₂R³⁴.

In another embodiment, in formula (IV), W represents a 5-membered heterocyclic ring comprising at least one ring heteroatom selected from nitrogen, oxygen and sulphur, wherein at least one of the ring carbon atoms may be optionally replaced by a carbonyl group; and wherein the heterocyclic ring is optionally substituted by at least one substituent selected from halogen, C₁-C₄ alkyl, C₁-C₄ alkoxy, CN, OH, NO₂, C₁-C₃ alkyl substituted by one or more F atoms, C₁-C₃ alkoxy substituted by one or more F atoms, NR⁴⁰R⁴¹, C≡CR⁴⁵, CONR⁴⁶R⁴⁷, CHO, C₂-C₄ alkanoyl, S(O)ₛR⁴⁸ and OSO₂R⁴⁹; and Z represents a single bond; and R¹⁴ represents phenyl or a 6-membered heteroaromatic ring comprising 1 to 3 ring nitrogen atoms; said ring being optionally substituted with at least one substituent selected from halogen, C₁-C₄ alkyl, C₁-C₄ alkoxy, CN, OH, NO₂, C₁-C₃ alkyl substituted by one or more F atoms, C₁-C₃ alkoxy substituted by one or more F atoms, NR¹²R¹³, C≡CR³⁰, CONR³¹R³², CHO, C₂-C₄ alkanoyl, S(O)ₚR³³ and OSO₂R³⁴;

In one embodiment R⁶ is a haloalkyl group.

In one embodiment, Y¹ to Y⁵ are each carbon atoms.

In one embodiment, A in formula (IIA) or (IIB) is a phenyl ring; D in formula (IIA) or (IIB) is an oxygen atom; and Y¹ to Y⁵ are each carbon atoms.

In one embodiment, W in formula (IV) represents S(O).

In an embodiment of the invention, Z represents a single bond, —CH₂—, —NH— or —NCH₃—. In another embodiment, Z represents a single bond such that the group W is bonded directly to the group R¹⁴.

Examples of saturated or unsaturated 3- to 10-membered ring systems that may be used, which may be monocyclic or polycyclic (e.g. bicyclic) in which the two or more rings are fused, include one or more (in any combination) of cyclopropyl, cyclobutyl, cyclopentyl, cyclohexyl, bicyclo[2.2.1]heptyl, cyclopentenyl, cyclohexenyl, phenyl, pyrrolidinyl, piperidinyl, piperazinyl, morpholinyl, thiomorpholinyl, diazabicyclo[2.2.1]hept-2-yl, naphthyl, benzofuranyl, benzothienyl, benzodioxolyl, quinolinyl, oxazolyl, 2,3-dihydrobenzofuranyl, tetrahydropyranyl, pyrazolyl, pyrazinyl, thiazolidinyl, indanyl, thienyl, isoxazolyl, pyridazinyl, thiadiazolyl, pyrrolyl, furanyl, thiazolyl, indolyl, imidazolyl, pyrimidinyl, benzimidazolyl, triazolyl, tetrazolyl and pyridinyl. Preferred ring systems include cyclopropyl, isoxazolyl and pyrazolyl.

In one embodiment of the invention, R¹⁴ represents phenyl or a 5- or 6-membered heteroaromatic ring system comprising one to three ring heteroatoms independently selected from nitrogen, oxygen and sulphur; each ring being optionally substituted by one or two substituents independently selected from F, Cl, Br, cyano, nitro, CF₃ and C≡CH.

Examples of a 5- or 6-membered heteroaromatic ring include furanyl, thienyl, pyrrolyl, oxazolyl, 1,2,4-oxadiazolyl, 1,3,4-oxadiazolyl, isoxazolyl, imidazolyl, pyrazolyl, thiazolyl, triazolyl, tetrazolyl, thiadiazolyl, pyridinyl, pyrimidinyl and pyrazinyl. Preferred heteroaromatic rings include thienyl, imidazolyl, pyridinyl, pyrimidinyl and pyrazinyl, especially pyridinyl.

In a further embodiment of the invention, R¹⁴ represents phenyl optionally substituted by one or two substituents independently selected from F, Cl, Br, cyano, nitro, CF₃ and C≡CH.

In one embodiment, R¹⁰ represents H.

In one embodiment, R⁸ represents a phenyl or pyridinyl ring substituted with at least one substituent (e.g. one, two or three substituents) independently selected from halogen (e.g. fluorine, chlorine, bromine or iodine), cyano, nitro, methyl, trifluoromethyl or methylcarbonyl.

In one embodiment, R⁸ represents a phenyl group substituted with one or two substituents independently selected from fluorine, chlorine, cyano, nitro, trifluoromethyl or methylcarbonyl.

In another embodiment, R⁸ represents a phenyl group substituted with one or two substituents selected from fluorine, chlorine or trifluoromethyl.

In still another embodiment, R⁸ represents a phenyl group substituted with a trifluoromethyl substituent (preferably in the meta position).

In one embodiment, R⁹ represents hydrogen or C₁-C₄ alkyl optionally substituted with one or two substituents independently selected from hydroxyl and C₁-C₄ alkoxy.

In another embodiment, R⁹ represents hydrogen.

In an embodiment of the invention, the compound of formula (IV) is one wherein:
R⁷ represents methyl;
W represents S(O);
Z represents a single bond;
R¹⁴ represents phenyl optionally substituted by one or two substituents independently selected from cyano, F, Cl, Br, CF₃, NO₂ and —C≡CH;
R¹⁰ represents H;
R⁸ represents a phenyl group substituted with a trifluoromethyl substituent; and
R⁴ represents hydrogen.

In one embodiment, L¹ is a direct bond.

In a further embodiment, X is the radical —N(R²³)—R²²—N(R²⁴)(R²⁵)—R²²—N(R²³)—.

In another embodiment, X is the radical —N(R²³)—R²²—N(R²⁷)—C(≡NR²⁶)—N(R²⁸)—R²²—N(R²³)—.

In yet another embodiment, R⁵ is an alkyl group.

In yet another embodiment, R⁵ is a methyl group.

In one embodiment, R⁴ is alkoxycarbonyl.

In a further embodiment, R⁴ is alkoxycarbonyl wherein the alkoxy group is substituted with a hydroxyl group.

In a further embodiment, R⁴ is alkoxycarbonyl wherein the alkoxy group is substituted with an amino group.

In a further embodiment, R⁴ is alkoxycarbonyl wherein the alkoxy group is substituted with an ammonium group.

Particular values for the linker group L in compounds of formula (I) include:

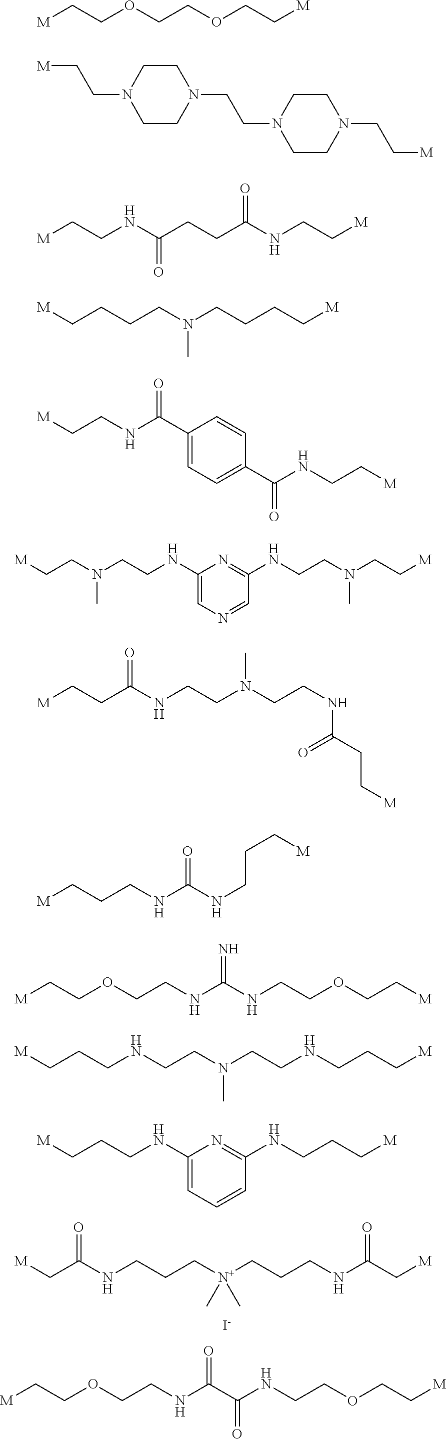

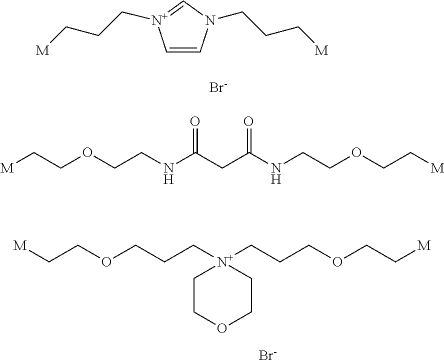

Particular values for the group M¹ in compounds of formula (I) include:

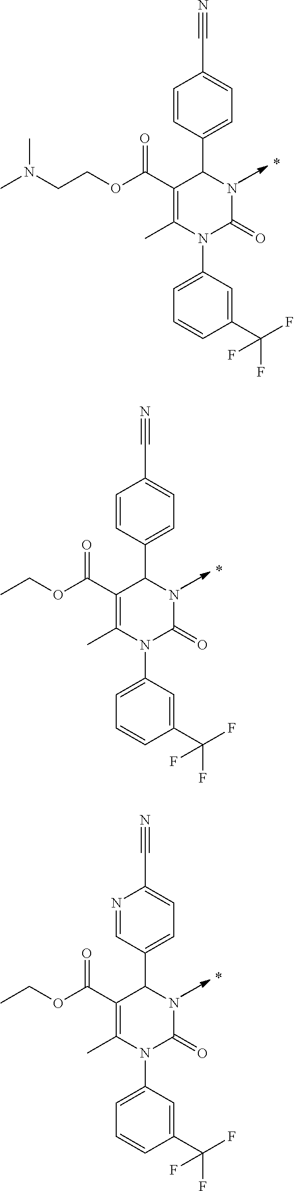

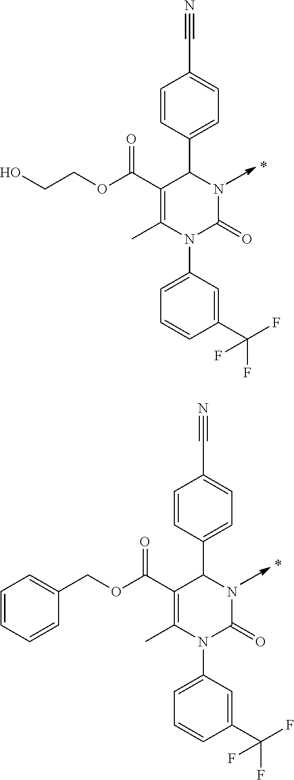

Particular values for the group M² in compounds of formula (I) include:

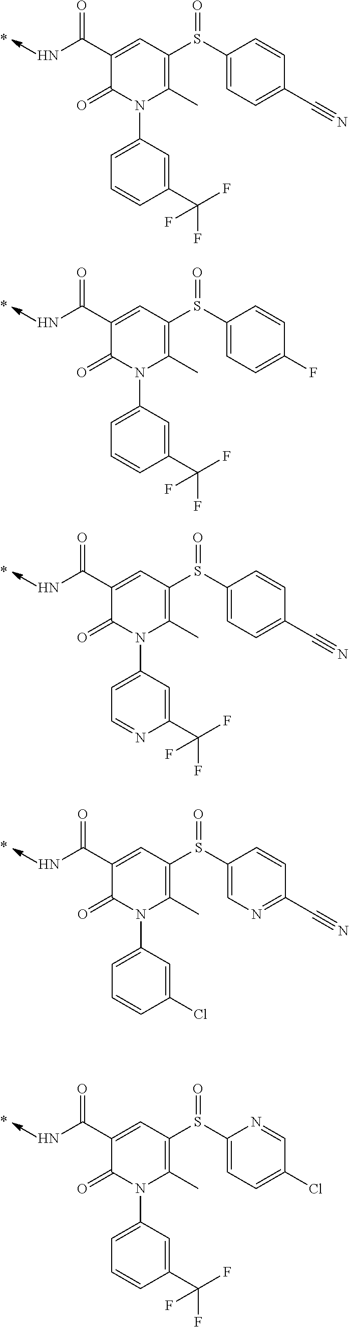

Particular compounds of the invention may be obtained by combining a particular linker group as illustrated above with a particular group M¹ as illustrated above and with a particular group M² as illustrated above.

Further particular compounds of the invention may be obtained by combining a particular linker group as illustrated above with two particular group M² as illustrated above, wherein the two M² groups may be the same or different.

In another aspect the present invention provides a compound of formula (VI):

[(M)-(L⁴)]ₜ-G (VI)

wherein:

t represents an integer 3 to 20;

L⁴ represents a linker group of formula -L¹-R¹⁵-L²-R¹⁶-L³-wherein L¹, L², L³, R¹⁵ and R¹⁶ are as defined above;

G represents is N, aryl, aryl¹-aryl², aryl¹-O-aryl², heteroaryl, heteroaryl¹-heteroaryl², heteroaryl¹-O-heteroaryl², a dendrimer or is selected from the following multivalent radicals wherein R²² is as defined above and r is an integer 1 to 6:

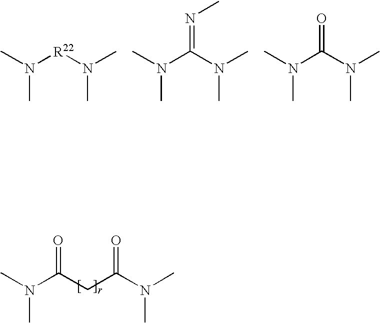

and M is as defined for formula (I) with the proviso that at least one M group represents M²;

or a pharmaceutically acceptable salt thereof.

Examples of group G include, but are not limited to, phenoxyphenyl, biphenyl, bipyridyl, ethylenediamino, propylenediamino and the like. It is to be understood that the number of possible attachment points is dictated by the valency of the groups present, so that for example, biphenyl can contain up to 10 possible attachments (5 on each phenyl ring), and ethylenediamine can possess up to 4 possible attachments (2 on each terminal amine). An example of a dendrimer suitable for use in the invention is:

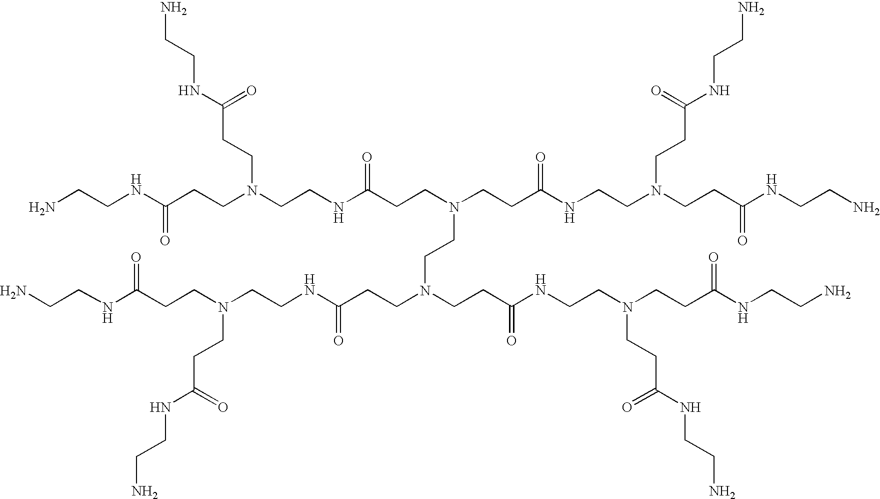

Preferred groups M¹ and M² for inclusion within the structures of compounds of formula (VI) include those specifically illustrated above.

In one embodiment, t represents an integer 3 to 5.

Particular structures for the compounds of formula (VI) include:

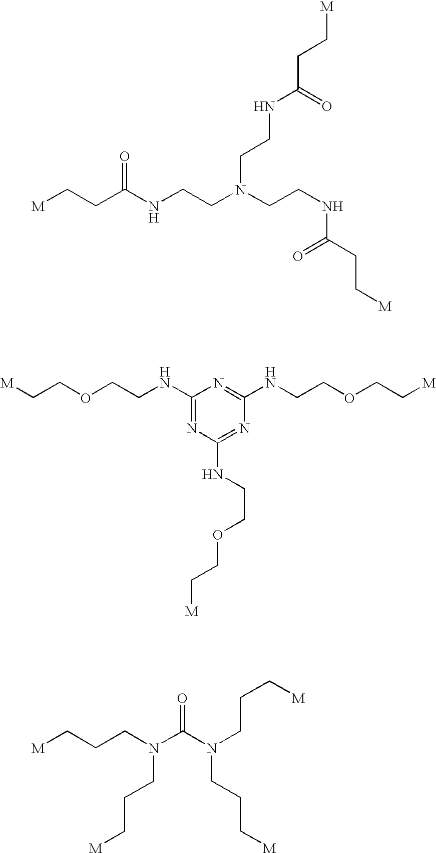

Examples of compounds of the invention include:

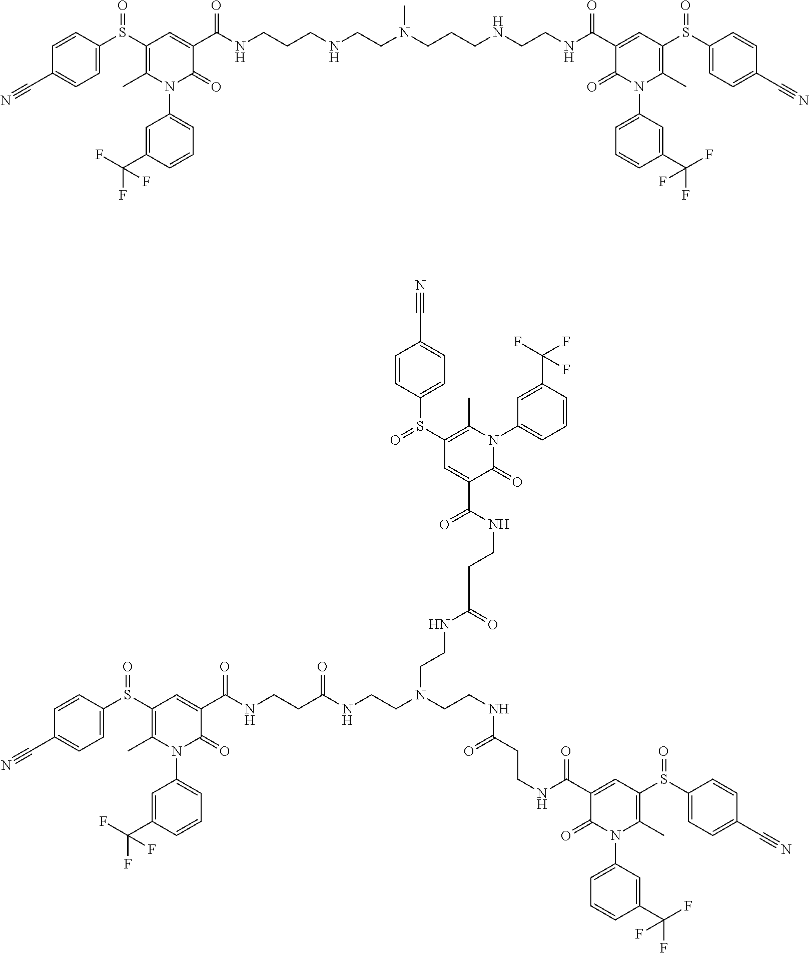

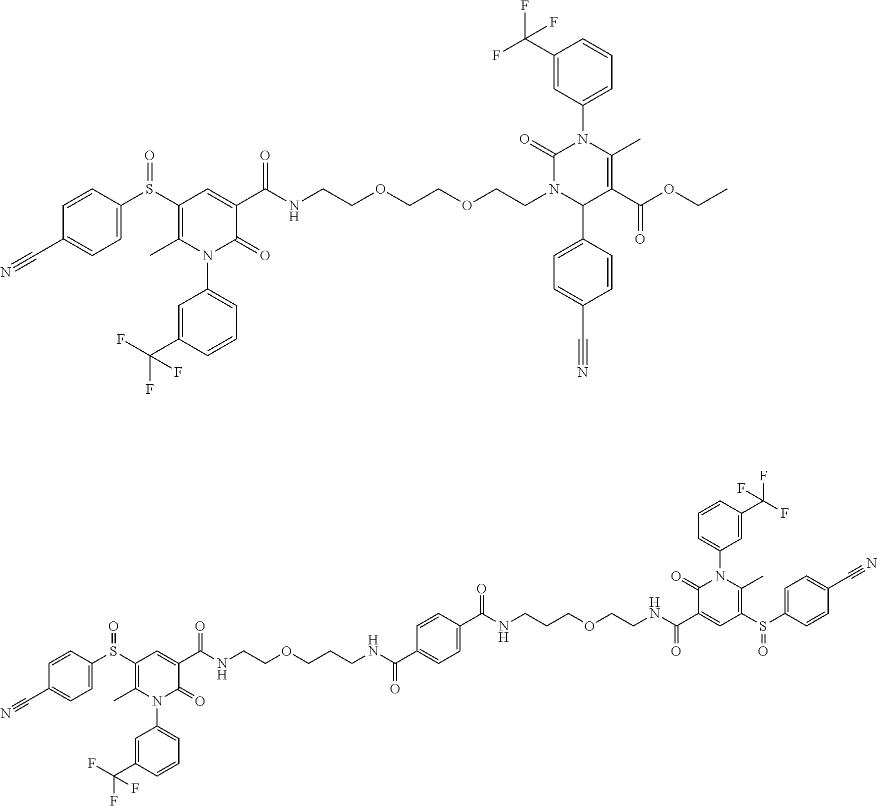

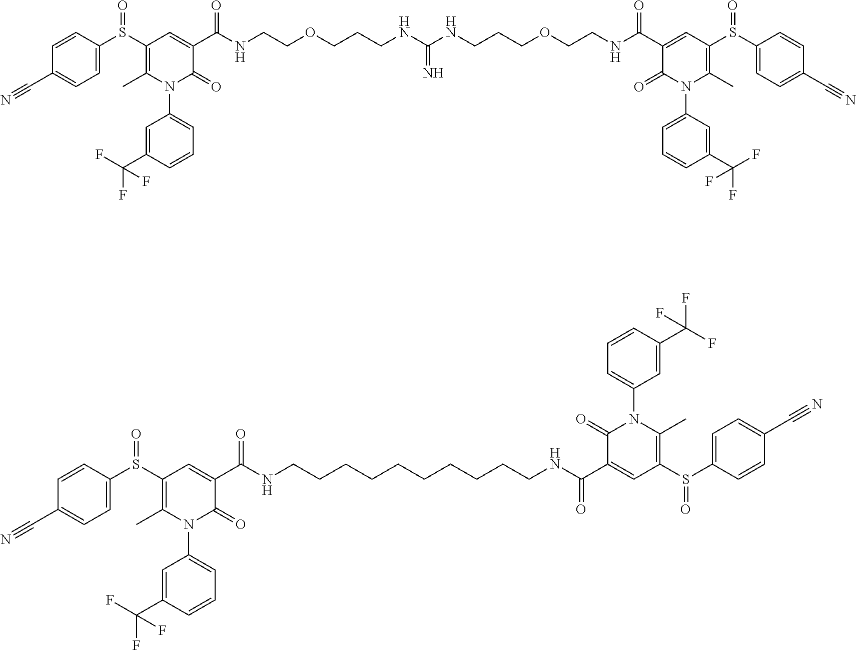

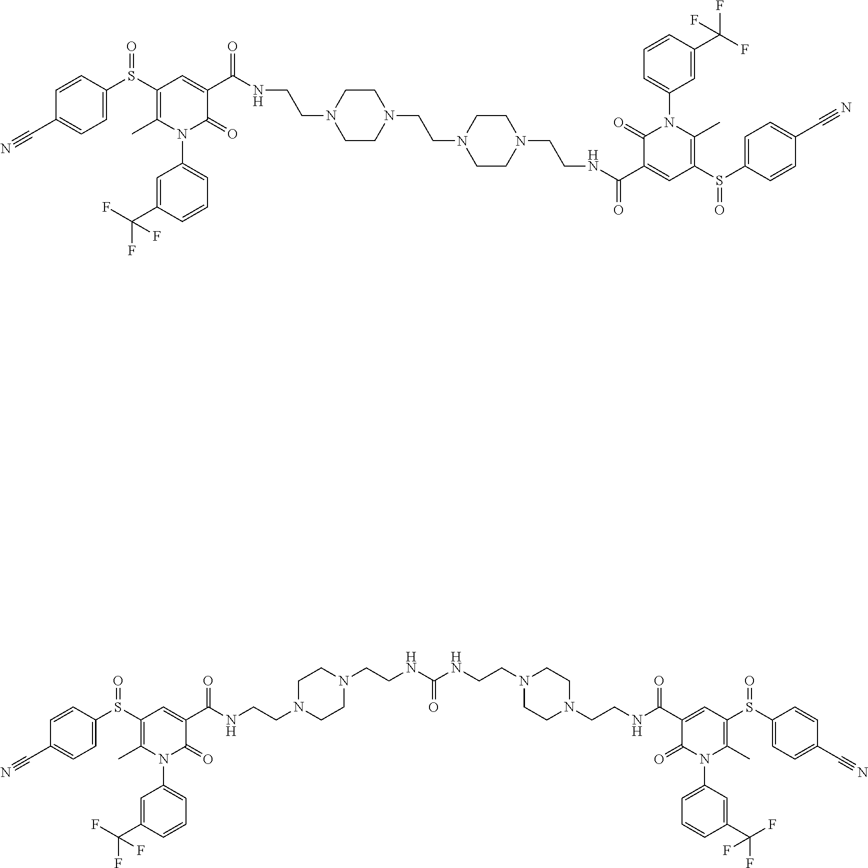

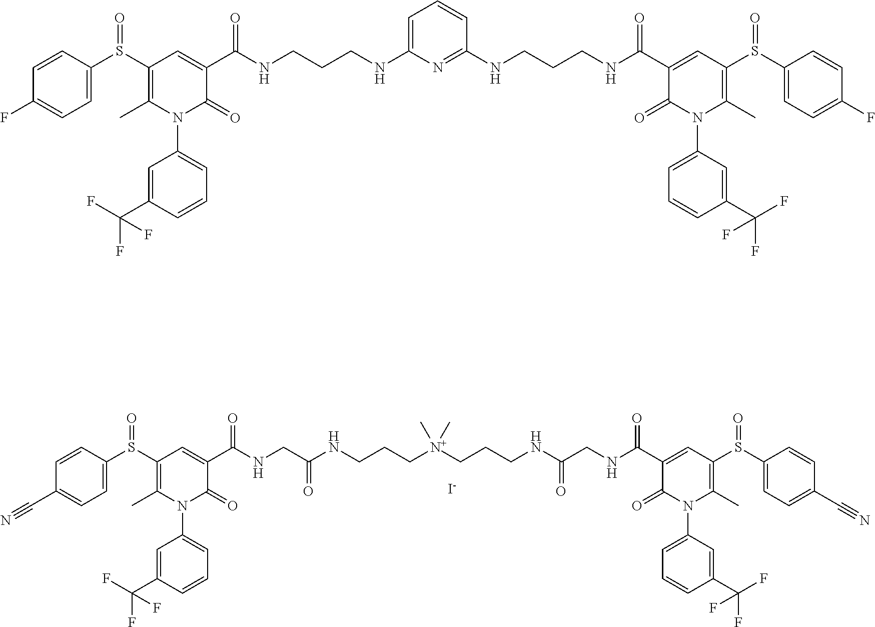

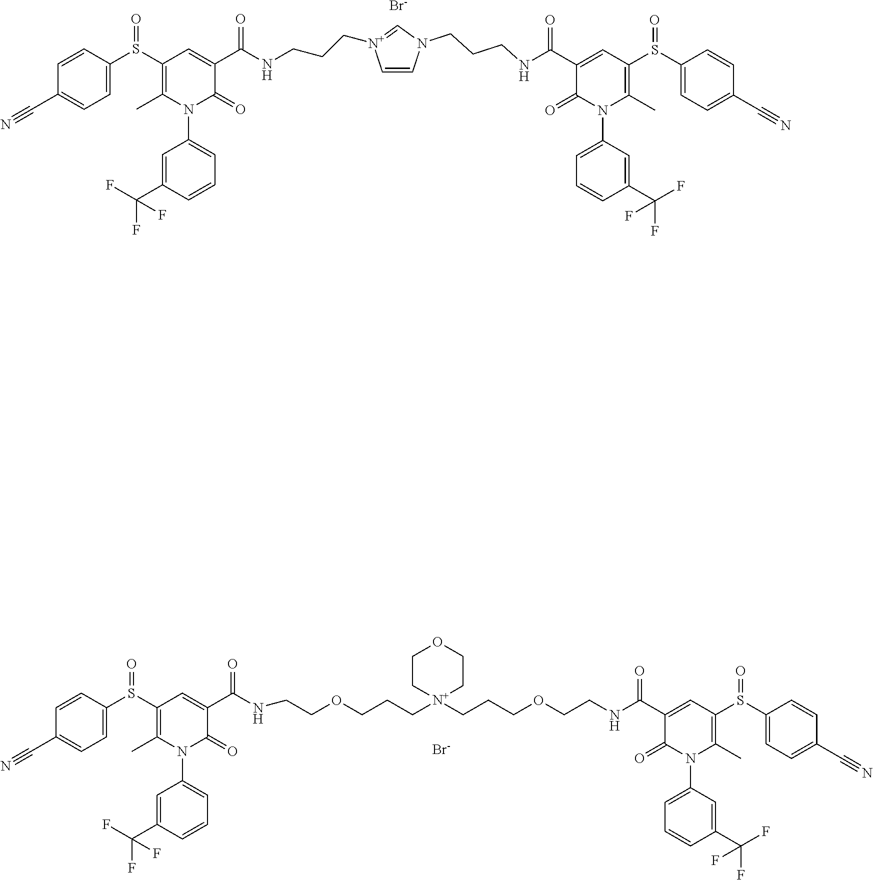

or a pharmaceutically acceptable salt of any one thereof.

The present invention further provides a process for the preparation of a compound of formula (I) or formula (VI) or a pharmaceutically acceptable salt thereof.

Processes for the preparation of compounds of formula (II) are disclosed in WO 2004/024700, WO 2004/024701, WO 2005/082863, WO 2005/082864 and GB 2 392 910.

Processes for the preparation of compounds of formula (IV) are disclosed in PCT/SE2006/000328.

Specific processes for the preparation of compounds of formula (I) are disclosed within the Examples section of the present specification. Such processes form an aspect of the present invention.

The necessary starting materials are either commercially available, are known in the literature or may be prepared using known techniques. Specific processes for the preparation of certain key starting materials are disclosed within the Examples section of the present specification and such processes form an aspect of the present invention.

It will be appreciated by those skilled in the art that in the processes of the present invention certain functional groups such as hydroxyl or amino groups may need to be protected by protecting groups. Thus, the preparation of the compounds of formula (I) or (VI) may involve, at an appropriate stage, the addition and/or removal of one or more protecting groups.

The protection and deprotection of functional groups is described in Protective Groups in Organic Chemistry', edited by J. W. F. McOmie, Plenum Press (1973) and ‘Protective Groups in Organic Synthesis’, 3^{rd} edition, T.W. Greene and P.G.M. Wuts, Wiley-Interscience (1999).

Typical processes are illustrated in the following schemes:

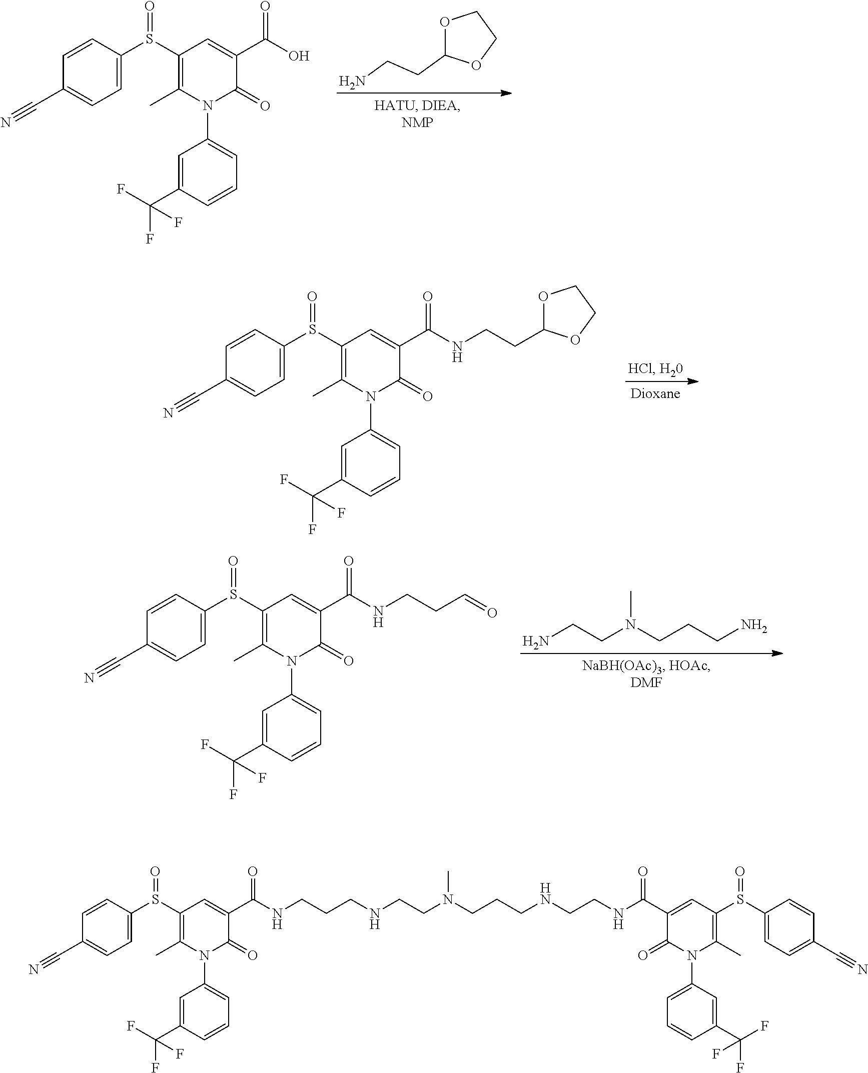

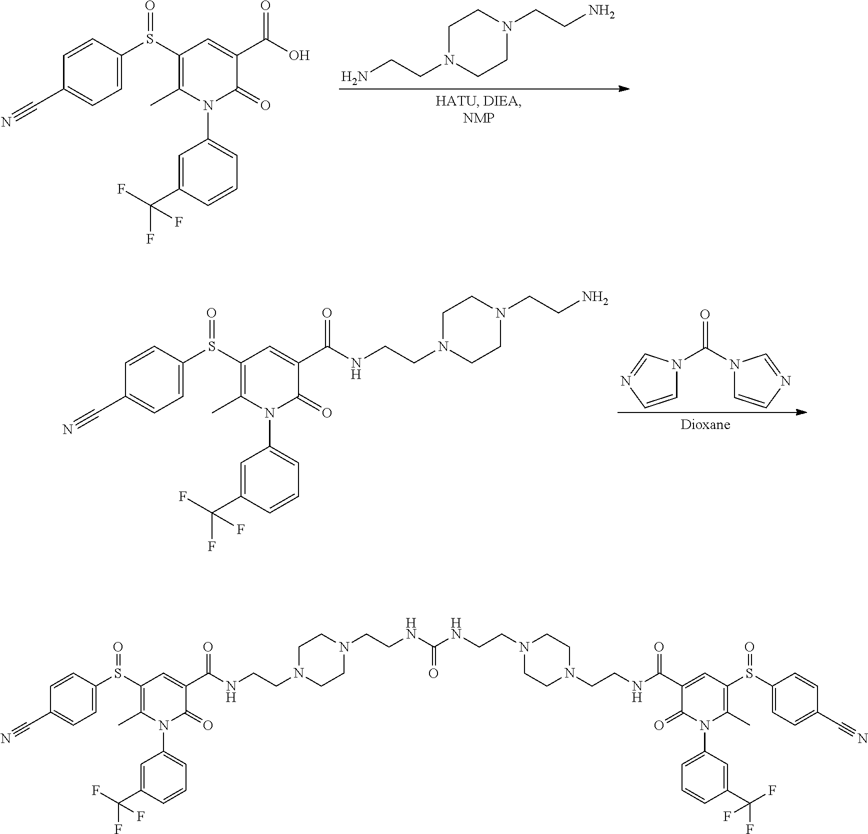

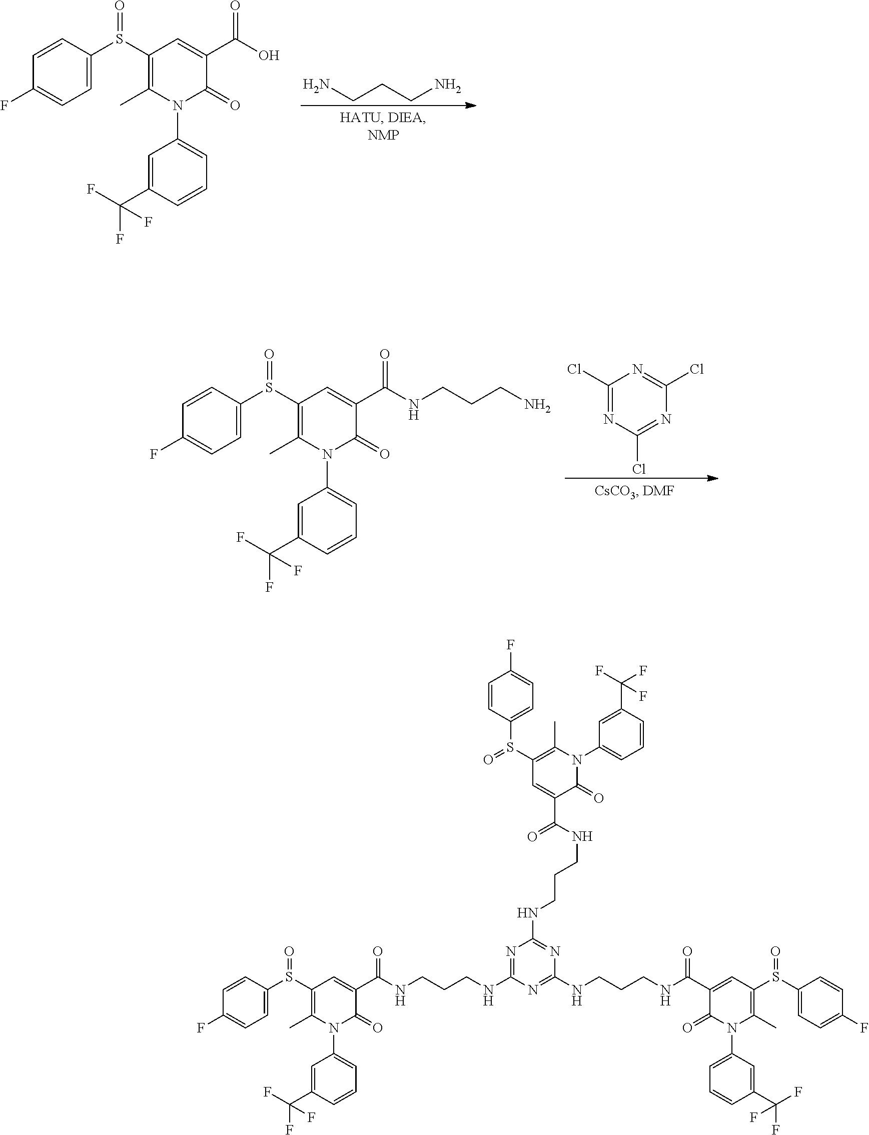

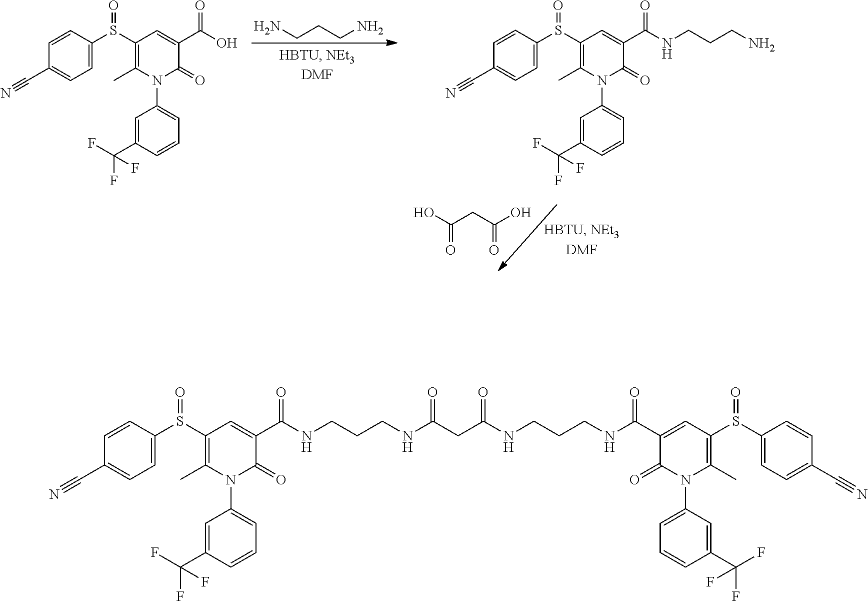

The compounds of formula (I) and formula (VI) may be converted to a pharmaceutically acceptable salt thereof, preferably an acid addition salt such as a hydrochloride, hydrobromide, sulphate, phosphate, acetate, fumarate, maleate, tartrate, lactate, citrate, pyruvate, succinate, oxalate, methanesulphonate or p-toluenesulphonate.

Compounds of formula (I) and formula (VI) are capable of existing in stereoisomeric forms. It will be understood that the invention encompasses the use of all geometric and optical isomers (including atropisomers) of the compounds of formula (I) and formula (VI) and mixtures thereof including racemates. The use of tautomers and mixtures thereof also form an aspect of the present invention. Enantiomerically pure forms are particularly desired.

The compounds of formula (I) and formula (VI) and their pharmaceutically acceptable salts have activity as pharmaceuticals, in particular as modulators of serine proteases such as proteinase 3 and pancreatic elastase and, especially, human neutrophil elastase, and may therefore be beneficial in the treatment or prophylaxis of inflammatory diseases and conditions.

Examples of such conditions include: adult respiratory distress syndrome (ARDS), cystic fibrosis, pulmonary emphysema, bronchitis, bronchiectasis, chronic obstructive pulmonary disease (COPD), systemic inflammatory response syndrome (SIRS) and ischaemic-reperfusion injury. The compounds of this invention may also be useful in the modulation of endogenous and/or exogenous biological irritants which cause and/or propagate atherosclerosis, diabetes, myocardial infarction; hepatic disorders including but not limited to cirrhosis, systemic lupus erythematous, inflammatory disease of lymphoid origin, including but not limited to T lymphocytes, B lymphocytes, thymocytes; autoimmune diseases, bone marrow; inflammation of the joint (especially rheumatoid arthritis, osteoarthritis and gout); inflammation of the gastro-intestinal tract (especially inflammatory bowel disease, ulcerative colitis, pancreatitis, peptic ulcers and gastritis); inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing and chronic wounds; demyelinating diseases of the central and peripheral nervous systems (such as multiple sclerosis); age related illness such as dementia, inflammatory diseases of cardiovascular origins; granulomatous diseases; renal diseases including but not limited to nephritis and polyarteritis; cancer; pulmonary hypertension, ingested poisons, skin contacts, stings, bites, sepsis; asthma; rhinitis; HIV disease progression; for minimising the effects of organ rejection in organ transplantation including but not limited to human organs; and replacement therapy of proteinase inhibitors.

Thus, the present invention provides a compound of formula (I) or a pharmaceutically-acceptable salt thereof as hereinbefore defined for use in therapy.

Thus, the present invention provides a compound of formula (VI) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In a further aspect, the present invention provides the use of a compound of formula (VI) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In a further aspect, the present invention provides the use of a compound of formula (I) or formula (VI) or a pharmaceutically acceptable salt thereof as hereinbefore defined in the manufacture of a medicament for the treatment of human diseases or conditions in which modulation of neutrophil elastase activity is beneficial.

In a further aspect, the present invention provides the use of a compound of formula (I) or formula (VI) or a pharmaceutically acceptable salt thereof as hereinbefore defined in the manufacture of a medicament for use in treating adult respiratory distress syndrome (ARDS), cystic fibrosis, pulmonary emphysema, bronchitis, bronchiectasis, chronic obstructive pulmonary disease (COPD), pulmonary hypertension, asthma, rhinitis, ischemia-reperfusion injury, rheumatoid arthritis, osteoarthritis, cancer, atherosclerosis or gastric mucosal injury.

The invention also provides a method of treating, or reducing the risk of, a disease or condition in which inhibition of neutrophil elastase activity is beneficial which comprises administering to a patient in need thereof a therapeutically effective amount of a compound of formula (I) or a pharmaceutically acceptable salt thereof as hereinbefore defined.

The invention still further provides a method of treating, or reducing the risk of, an inflammatory disease or condition which comprises administering to a patient in need thereof a therapeutically effective amount of a compound of formula (I) or a pharmaceutically acceptable salt thereof as hereinbefore defined.

The invention also provides a method of treating, or reducing the risk of, a disease or condition in which inhibition of neutrophil elastase activity is beneficial which comprises administering to a patient in need thereof a therapeutically effective amount of a compound of formula (VI) or a pharmaceutically acceptable salt thereof as hereinbefore defined.

The invention still further provides a method of treating, or reducing the risk of, an inflammatory disease or condition which comprises administering to a patient in need thereof a therapeutically effective amount of a compound of formula (VI) or a pharmaceutically acceptable salt thereof as hereinbefore defined.

In particular, the compounds of this invention may be used in the treatment of adult respiratory distress syndrome (ARDS), cystic fibrosis, pulmonary emphysema, bronchitis, bronchiectasis, chronic obstructive pulmonary disease (COPD), pulmonary hypertension, asthma, rhinitis, ischemia-reperfusion injury, rheumatoid arthritis, osteoarthritis, cancer, atherosclerosis or gastric mucosal injury.

For the above-mentioned therapeutic uses the dosage administered will, of course, vary with the compound employed, the mode of administration, the treatment desired and the disorder indicated. The daily dosage of the compound of the invention may be in the range from 0.05 mg/kg to 100 mg/kg.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The present invention also provides a pharmaceutical composition comprising a compound of formula (VI) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or formula (VI) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Inhalation is a preferred method of administration. Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and May be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

In particular the compounds of the invention may be administered in conjunction with a second active ingredient which is selected from:
a) a PDE4 inhibitor including an inhibitor of the isoform PDE4D;
b) a β-adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
c) a muscarinic receptor antagonist (for example a M1, M2 or M3 antagonist, such as a selective M3 antagonist) such as ipratropium bromide, tiotropium bromide, oxitropium bromide, pirenzepine or telenzepine;
d) a modulator of chemokine receptor function (such as a CCR1 or CCR8 receptor antagonist);
e) an inhibitor of p38 kinase function;
f) an IKK2 antagonist;
g) a glucocorticoid receptor ligand;
h) a glucocorticoid;
i) a statin;
j) a MMP inhibitor (such as a MMP12 or MMP9 inhibitor);
k) an epidermal growth factor inhibitor; and
l) a histamine type I receptor antagonist.

The present invention will now be further explained by reference to the following illustrative examples.

### Example 1

### N,N′-[Ethane-1,2-diylbis(oxyethane-2,1-diyl)]bis {5-[4-cyanophenyl)sulfinyl]-6-methyl-2-oxo-1-[3-(trifluoromethyl)phenyl]-1,2 dihydropyridine-3-carboxamide}

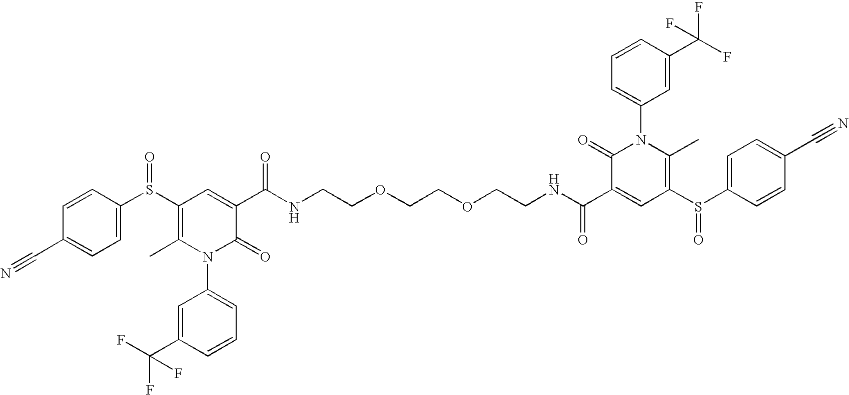

To a mixture of 5-[(4-cyanophenyl)sulfinyl]-6-methyl-2-oxo-1-[3-(trifluoromethyl)phenyl]-1,2-dihydropyridine-3-carboxylic acid (Intermediate 1, 38.3 mg, 0.086 mmol), HBTU (37 mg, 0.098 mmol) and triethylamine (100 μl, 0.72 mmol) in dry DMF (0.5 ml) was added 2.2-(ethylenedioxyl)diethylamine (5.8 μl, 0.039 mmol) after 5 minutes stirring at room temperature. The reaction was completed after 1 h according to LC-MS. The reaction mixture was diluted with acetonitrile /water and purified by preparative HPLC to give the title compound as a white powder (19 mg, 49%). ¹H NMR (500 MHz, DMSO-d₆) δ 9.13 (bt, J=5.17 Hz, 2H), 8.27 (d, J=2.87 Hz, 2H), 8.09-8.06 (m, 4H), 8.04-8.02 (m, 1H), 7.97 (bs, 1H), 7.91 (bd, J=7.73 Hz, 1H), 7.89-7.87 (m, 5H), 7.85-7.81 (m, 3H), 7.77 (bd, J=7.96 Hz, 1H), 3.40 (s, 4H), 3.39-3.34 (m, 4H), 3.30-3.24 (m, 4H), 2.34 (s, 6H).

APCI-MS ^{m}/z: 1005.6 (MH⁺).

### Example 2

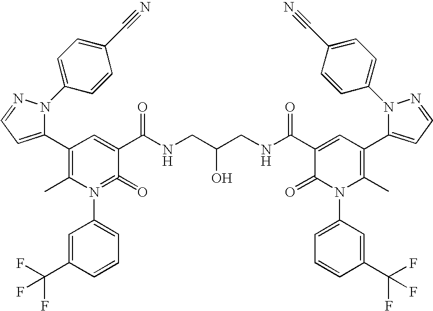

### N,N′-(2-Hydroxypropane-1,3-diyl)bis(5-(1-(4-cyanophenyl)-1H-pyrazol-5-yl)-6-methyl-2-oxo-1-(3-(trifluoromethyl)phenyl)-1,2-dihydropyridine-3-carboxamide)

5-(1-(4-Cyanophenyl)-1H-pyrazol-5-yl)-6-methyl-2-oxo-1-(3-(trifluoromethyl)phenyl)-1,2-dihydropyridine-3-carboxylic acid (36 mg, 0.08 mmol), HBTU (38.2 mg, 0.10 mmol) and DIEA (0.093 mL, 0.23 mmol) were dissolved in NMP (15 mL) and stirred for 30 minutes at room temperature before 1,3-diamino-2-propanol (6.99 mg, 0.08 mmol) was added. The final reaction mixture was stirred overnight at room temperature. The reaction mixture was partitioned between EtOAc and water. The organic layer was washed with water, dried and evaporated. Purification on preparative HPLC and freeze drying gave 17 mg (22% yield) of the title compound.

¹H NMR (300 MHz, CD₃CN) δ 9.59 (bt, 2H), 8.25 (s, 2H), 7.87-7.73 (m, 10H), 7.63-7.49 (m, 8H), 6.60 (d, 2H), 3.95 (d, 1H), 3.83-3.72 (m, 1H), 3.49-3.23 (dm, 4H), 1.68 (s, 6H).

APCI-MS ^{m}/z: 984.1 (MH⁺).

### Intermediate 1

### 5-[(4-Cyanophenyl)sulfinyl]-6-methyl-2-oxo-1-[3-(trifluoromethyl)phenyl]-1,2-dihydropyridine-3-carboxylic acid

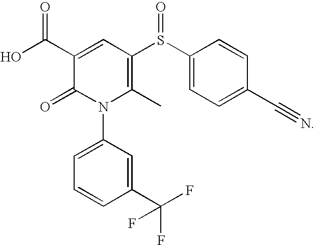

### a) Ethyl 5-[(4-cyanophenyl)sulfinyl]-6-methyl-2-oxo-1-[3-(trifluoromethyl)phenyl]-1,2-dihydropyridine-3-carboxylate

4-Mercaptobenzonitrile (200 mg, 1.5 mmol) and ethyl 5-iodo-6-methyl-2-oxo-1-[3-(trifluoromethyl)phenyl]-1,2-dihydropyridine-3-carboxylate (prepared as described in WO 2005/026123; 225 mg, 0.5 mmol) were mixed in DMF (8 ml). Bis(tri-t-butylphosphine)palladium (0.1 eq.) was added and the mixture degassed by bubbling argon through the solution (3 min), whereupon the mixture was heated in a microwave oven to 150° C. for 30 min. The reaction mixture was partitioned between EtOAc and brine. The organic phase was evaporated and the residue was re-dissolved in EtOAc and filtered through a short column of silica. Evaporation of the solvent, purification by HPLC, and freeze-drying afforded the sulfide as an amorphous solid. The sulfide was dissolved in acetic acid (10 ml), hydrogen peroxide (2 ml of a 35% aqueous solution) was added and the resulting mixture was heated at 50° C. for 40 min. to obtain the title sulfoxide, which was purified by HPLC (8 mg).

¹H NMR (399.99 MHz, DMSO-d₆) δ 8.13-7.68 (m, 9H), 4.18 (q, J=7.2 Hz, 2H), 2.29 (s, 3H), 1.19 (t, J=7.1 Hz, 3H).

APCI-MS ^{m}/z: 475.0 (MH⁺).

### b) 5-[(4-Cyanophenyl)sulfinyl]-6-methyl-2-oxo-1-[3-(trifluoromethyl)phenyl]-1,2-dihydropyridine-3-carboxylic acid

To ethyl 5-[(4-cyanophenyl)sulfinyl]-6-methyl-2-oxo-1-[3-(trifluoromethyl)phenyl]-1,2-dihydropyridine-3-carboxylate (80 mg, 0.17mmol) in dioxane (5 ml), H₂SO₄ (1 ml) and water (0.5 ml) were added. The mixture was heated to 80° C. for 2 hours and then purged on ice water. After extraction with ethyl acetate (3×10 ml), the organic phase was dried (MgSO₄) and evaporated. The residue was dissolved in acetic acid and freeze dried affording the title compound (70 mg, 93%).

¹H NMR (399.99 MHz, DMSO-d₆) δ 13.25 (bs, 1H), 8.24 (d, J=3.41 Hz, 1H), 8.11 (dd, J=8.41 1.57 Hz, 2H), 8.01 (bd, J=5.37 Hz, 1H), 7.96 (bd, J=7.89 Hz, 1H), 7.93 (d, J=8.35 Hz, 2H), 7.90-7.80 (m, 2H), 2.35 (s, 3H).

APCI-MS ^{m}/z: 447.2 (MH⁺).

### Intermediate 2

### a) Ethyl 5-iodo-6-methyl-2-oxo-1-(3-trifluoromethylphenyl)-1,2-dihydro-pyridine-3-carboxylate

5-Iodo-6-methyl-2-oxo-1-(3-trifluoromethylphenyl)-1,2-dihydropyridine-3-carboxylic acid (191 mg, 0.45 mmol) was dissolved in dichloromethane (2 ml) and thionyl chloride (5 ml, 68 mmol) was added. The solution was stirred at room temperature for one hour. The solvents were evaporated off and the residue was dissolved in dichloromethane (3 ml) and EtOH (99%) (15 ml) was added. The solution was stirred at room temperature for two hours. The solvents were evaporated to give crude product (200 mg, 98% yield), which was used in the next step without further purification.

¹H NMR (400 MHz, aceton-d₆) δ 8.35 (s, 1H), 7.87-7.80 (m, 3H), 7.71-7.67 (m, 1H), 4.25 (q, 2H), 2.27 (s, 3H), 1.29 (t, 3H).

APCI-MS ^{m}/z: 451.9 (MH⁺).

### b) Ethyl 5-(3,3-diethoxy-prop-1-ynyl)-6-methyl-2-oxo-1-(3-trifluoromethylphenyl)-1,2-dihydro-pyridine-3-carboxylate

Ethyl 5-iodo-6-methyl-2-oxo-1-(3-trifluoromethyl-phenyl)-1,2-dihydropyridine-3-carboxylate (200 mg, 0.44 mmol), propargylaldehyde diethyl acetal (100 μl, 0.69 mmol), bis(triphenylphosphine)-palladium(II)chloride (9.8 mg, 0.014 mmol), copper(I)iodide (5.8 mg, 0.03 mmol) and triethylamine (1.5 ml) were mixed in THF (2 ml). The mixture was degassed with argon and then heated in a 150 W microwave at 90° C. for 10 minutes. The solution was filtered through a plug of silica with EtOAc as eluent. Purification on preparative HPLC and freeze drying gave 142 mg (70% yield) of the title compound.

¹H NMR (300 MHz, aceton-d₆) δ 9.09 (bs, 1H), 8.41 (s, 1H), 7.96-7.85 (m, 3H), 7.80-7.75 (m, 1H), 5.52 (s, 1H), 3.82-3.57 (m, 4H), 2.87 (d, 3H), 2.26 (s, 3H), 1.20 (t, 6H).

APCI-MS ^{m}/z: 452.0 (MH⁺).

### c) Ethyl 5-[2-(4-cyanophenyl)-2H-pyrazole-3-yl]-6-methyl-2-oxo-1-(3-trifluoromethylphenyl)-1,2-dihydropyridine-3-carboxylate

Ethyl 5-(3,3-diethoxy-prop-1-ynyl)-6-methyl-2-oxo-1-(3-trifluoromethylphenyl)-1,2-dihydropyridine-3-carboxylate (957 mg, 2.12 mmol) and 4-cyanophenylhydrazine hydrochloride (565 mg, 3.33 mmol) were dissolved in EtOH (99%) (3 mL). The mixture was heated in a 150 W microwave at 120° C. for 30 minutes. Purification on preparative HPLC and freeze drying gave 920 mg (88% yield) of the title compound.

¹H NMR (400 MHz, DMSO-d₆) δ 7.98-7.77 (m, 7H), 7.69-7.63 (m, 3H), 6.71 (d, 1H), 4.14 (q, 2H), 1.69 (s, 31.1), 1.17 (t, 3H).

APCI-MS ^{m}/z: 493.0 (MH⁺).

### d) 5-[2-(4-Cyanophenyl)-2H-pyrazol-3-yl]-6-methyl-2-oxo-1-(3-trifluoromethyl-phenyl)-1,2-dihydropyridine-3-carboxylic acid

Ethyl 5-[2-(4-cyanophenyl)-2H-pyrazol-3-yl]-6-methyl-2-oxo-1-(3-trifluoromethyl-phenyl)-1,2-dihydropyridine-3-carboxylate (118 mg, 0.24 mmol) was dissolved in THF (3 ml). LiOH (2M, aq) (3 ml) was added and the mixture was stirred overnight at room temperature. The THF was evaporated off, and EtOAc and water were added to the water containing residue. The mixture was acidified to pH3 with 1M aqueous hydrochloric acid. The phases were separated and the organic layer was washed with water, dried (MgSO₄), filtered and evaporated. Purification on preparative HPLC and freeze drying gave 56 mg (50% yield) of the desired product.

¹H NMR (400 MHz, CD₃CN) δ 8.30 (s, 1H), 7.93-7.77 (m, 5H), 7.68-7.56 (m, 4H), 6.65 (d, 1H), 1.79 (s, 3H).

APCI-MS ^{m}/z: 464.9 (MH⁺).

Human Neutrophil Elastase Quenched-FRET Assay

The assay uses Human Neutrophil Elastase (HNE) purified from serum (Calbiochem art. 324681; Ref. Baugh, R. J. et al., 1976, Biochemistry. 15, 836-841). HNE was stored in 50 mM sodium acetate (NaOAc), 200 mM sodium chloride (NaCl), pH 5.5 with added 30% glycerol at −20° C. The protease substrate used was Elastase Substrate V Fluorogenic, MeOSuc-AAPV-AMC (Calbiochem art. 324740; Ref. Castillo, M. J. et al., 1979, Anal. Biochem. 99, 53-64). The substrate was stored in dimethyl sulphoxide (DMSO) at −20° C. The assay additions were as follows: Test compounds and controls were added to black 96-well flat-bottom plates (Greiner 655076), 1 μL in 100% DMSO, followed by 30 μL HNE in assay buffer with 0.01% Triton (trade mark) X-100 detergent. The assay buffer constitution was: 100 mM Tris(hydroxymethyl)aminomethane (TRIS) (pH 7.5) and 500 mM NaCl. The enzyme and the compounds were incubated at room temperature for 15 minutes. Then 30 μl substrate in assay buffer was added. The assay was incubated for 30 minutes at room temperature. The concentrations of HNE enzyme and substrate during the incubation were 1.7 nM and 100 μM, respectively. The assay was then stopped by adding 60 μl stop solution (140 mM acetic acid, 200 mM sodium monochloroacetate, 60 mM sodium acetate, pH 4.3). Fluorescence was measured on a Wallac 1420 Victor 2 instrument at settings: Excitation 380 nm, Emission 460 nm. IC₅₀ values were determined using Xlfit curve fitting using model 205.

When tested using the above assay the compounds of the Examples were shown to have desirable HNE inhibitory activity (Table).

TABLE

| Compound | Inhibition of HNE IC₅₀ (nM) |
|---|---|
| Example 1 | 0.61 |
| Example 2 | 0.26 |

Human Neutrophil Elastase Induced Lung Haemorrhage in the Rat

Instillation of human neutrophil elastase (HNE) into rat lungs causes acute lung injury (ALI). Measuring lung haemorrhage can assess the extent of this injury. Female Wistar rats (180-220 g) were obtained from Taconic M&B, Denmark, barrier bred and certified free from specified microorganisms. Animals were weighed and randomly assigned to treatment groups (5-15 animals per group). Animals in each study used to determine the efficacy of the elastase inhibitors delivered locally to the lung by a variety of routes. Rats were anaesthetised with inhaled Isoflurane (2-5%) when the dose was given from 30 minutes to 1 hr prior to HNE administration. The animals were than either dosed intratracheally (i.t.) using a modified, angled metal cannula or intranasally (i.n.) by dropping the fluid on the nares. Animals either received vehicle or compound at a dose volume of 1.0 ml/kg (i.t.) or 0.25 ml/kg (i.n.). The vehicle used for the inhibitors was a Polysorbate 80 vehicle. Animals were than anaesthetised with inhaled Isoflurane (2-5%) and the i.t. instillation of HNE (250 units/ml) or sterile saline was administered by a modified, angled metal cannula at a volume of 200 μl/animal. The animals were then kept in their regular cage, moving about freely until termination. Animals were sacrificed (1-2 ml sodium pentobarbitone 60 mg/ml, i.p) 4 hour post HNE challenge. The trachea was exposed and a small incision made between two cartilage rings, just below larynx allowing a catheter to be inserted approximately 1 cm into the trachea towards the lung and secured with a suture. The catheter was assembled with a syringe connector and bronchoalveolar lavage tube to a reservoir (15 cm H₂O). The lungs were then lavaged twice with fresh phosphate buffered saline (PBS). The lavage fluid was kept on ice until it was cetrifugated. The bronchoalveolar lavage fluid (BAL) was centrifugated at 1200 r.p.m. at 4° C. for 15 minutes. The supernatant was collected and the cell pellet was lysated with 3 ml distilled water. A standard curve was made from stock solution of lysated blood cells (2000 μg/ml). 150 μl of standards and BAL samples in duplicate were transferred into a 96-well plate and OD was measured at 412 nm using a Spectramax 340PC. The amount of haemoglobin in each BALsample was calculated by comparison to the standard curve (31, 62, 125, 250, 500, 100 μg/ml). The mean OD for duplicates was calculated and expressed as mean haemoglobin±standard error of the mean (SEM). The compounds were shown to have desirable HNE inhibitory activity.

Lipopolysaccharide (LPS)-Induced Acute Lung Inflammation in the Rat

Female Wistar rats (180-220 g) were obtained from Taconic M&B, Denmark, barrier bred and certified free from specified microorganisms. Animals were weighed and randomly assigned to treatment groups (5-15 animals per group). Animals in each study used to determine the efficacy of the elastase inhibitors delivered locally to the lung by a variety of routes. Rats were anaesthetised with inhaled Isoflurane (2-5%) when the dose was given from 60 minutes to LPS administration. The animals were either dosed intratracheally (i.t.) using a modified, angled metal cannula or intranasally (i.n.) by dropping the fluid on the nares. Animals either received vehicle or compound at a dose volume of 1.0 ml/kg (i.t.) or 0.25 ml/kg (i.n.). The vehicle used for the inhibitors was a Polysorbate 80 vehicle. Animals were anaesthetised with inhaled Isoflurane (2-5%) and the i.t. instillation of LPS (10 μg/ml/rat, E.coli 026:B6, Sigma-Aldrich) or sterile saline was administered by a modified, angled metal cannula at a volume of 200 μl/animal. The animals were then kept in their regular cage, moving about freely until termination. Animals were sacrificed (1-2 ml sodium pentobarbitone 60 mg/ml, i.p.) 4 hour post LPS challenge. The trachea was exposed and a small incision made between two cartilage rings, just below larynx allowing a catheter to be inserted approximately 1 cm into the trachea towards the lung and secured with a suture. The catheter was assembled with a syringe connector and lavage tube to a reservoir at 15 cmH₂O. The lungs were then lavaged two times with fresh phosphate buffered saline (PBS). The lavage fluid was kept on ice until it was cetrifugated. The bronchoalveolar lavage fluid (BAL) was centrifugated at 1200 r.p.m. at 4° C. for 15 minutes. The BAL supernatant were collected and rat neutrophil elastase activity was measured using fluorgenic neutrophil elastase substrate. The compounds were shown to inhibit rat neutrophil elastase activity in BAL.

1. A compound of formula (I)
(M)-(L)-(M) (I)
wherein:
either M represents a group M¹ of formula (IIA) or (IIB): 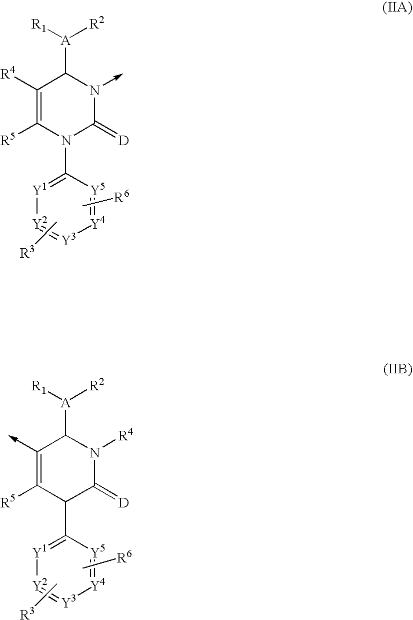
wherein:
A is aryl or heteroaryl;
D is oxygen or sulphur;
R¹, R² and R³ are each independently hydrogen, halogen, nitro, cyano, alkyl, hydroxy or alkoxy; wherein said alkyl and alkoxy may be further substituted with one to three identical or different radicals selected from the group consisting of halogen, hydroxy and alkoxy;
R⁴ is hydrogen, alkyl, trifluoromethylcarbonyl, alkylcarbonyl, alkoxycarbonyl, alkenoxycarbonyl, hydroxycarbonyl, aminocarbonyl, arylcarbonyl, heteroarylcarbonyl, heterocycloalkylcarbonyl, heteroaryl, heterocycloalkyl or cyano; wherein said alkylcarbonyl, alkoxycarbonyl and aminocarbonyl may be further substituted with one to three identical or different radicals selected from the group consisting of cycloalkyl, hydroxy, alkoxy, alkoxycarbonyl, hydroxycarbonyl, aminocarbonyl, cyano, amino, heteroaryl, heterocycloalkyl and tri-(alkyl)-silyl; and
wherein said heteroarylcarbonyl, heterocycloalkylcarbonyl, heteroaryl and heterocycloalkyl may be further substituted with alkyl;
or
R⁴ represents a group of Formula (III): 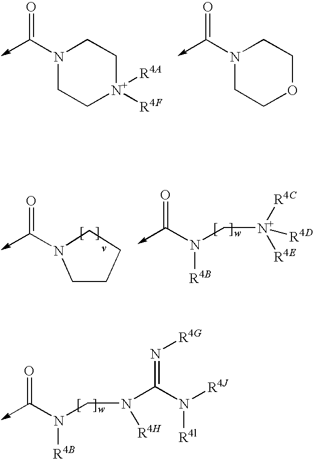
wherein
R^{4A}, R^{4B}, R^{4G}, R^{4H}, R^{4I} and R^{4J} are each independently hydrogen or alkyl; or R^{4H} and R^{4I} may be joined together with the nitrogen atoms to which they are attached to form a ring;
R^{4F} is a lone pair or R^{4F} is alkyl and the nitrogen atom to which it is attached is quaternary and carries a positive charge;
R^{4C}, R^{4D} and R^{4E} are alkyl, or any two of R^{4C}, R^{4D} and R^{4E} may be joined together with the nitrogen atom to which they are attached to form a ring, optionally containing a further heteroatom selected from oxygen or nitrogen;
v is an integer 1 to 3;
W is an integer 1 to 6;
R⁵ is alkyl, which may be optionally substituted with one to three identical or different radicals selected from the group consisting of halogen, hydroxy, alkoxy, alkenoxy, alkylthio, amino, hydroxycarbonyl, alkoxycarbonyl and the radical —O-(alkyl)-O-(alkyl);
or R⁵ is amino;
R⁶ is halogen, nitro, cyano, alkyl, hydroxy or alkoxy; wherein said alkyl and alkoxy may be further substituted with one to three identical or different radicals selected from the group consisting of halogen, hydroxy and alkoxy;
Y¹, Y², Y³, Y⁴ and Y⁵ are each independently C or N, with the proviso that the ring in which they are comprised contains no more than two N atoms; and
→ indicates the preferred point of attachment of M¹ to the group L;
or M represents a group M² of formula (IV): 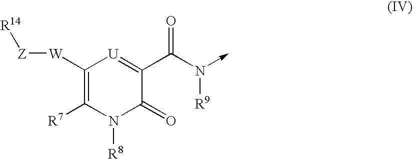
wherein
R⁷ represents hydrogen or alkyl;
U represents N or CR¹⁰;
Either W represents S(O)ₘ wherein m represents an integer 0, 1 or 2; and
Z represents a single bond, —CH₂— or —NR³⁷—; and
R¹⁴ represents a hydrogen atom or OH or a group selected from alkyl and a saturated or unsaturated 3- to 10-membered ring system optionally comprising at least one ring heteroatom selected from nitrogen, oxygen and sulphur; each group being optionally substituted with at least one substituent selected from phenyl, alkoxycarbonyl, halogen, alkyl, alkoxy, CN, OH, NO₂, alkyl substituted by one or more F atoms, alkoxy substituted by one or more F atoms, NR¹²R¹³, C≡CR³⁰, CONR³¹R³², CHO, alkylcarbonyl, S(O)ₚR³³ and OSO₂R³⁴;
Or W represents a 5-membered heterocyclic ring comprising at least one ring heteroatom selected from nitrogen, oxygen and sulphur, wherein at least one of the ring carbon atoms may be optionally replaced by a carbonyl group; and wherein the heterocyclic ring is optionally substituted by at least one substituent selected from halogen, C₁-C₄ alkyl, C₁-C₄ alkoxy, CN, OH, NO₂, C₁-C₃ alkyl substituted by one or more F atoms, C₁-C₃ alkoxy substituted by one or more F atoms, NR⁴⁰R⁴¹, C≡CR⁴⁵, CONR⁴⁶R⁴⁷, CHO, C₂-C₄ alkanoyl, S(O)ₛR⁴⁸ and OSO₂R⁴⁹; and
Z represents a single bond; and
R¹⁴ represents phenyl or a 6-membered heteroaromatic ring comprising 1 to 3 ring nitrogen atoms; said ring being optionally substituted with at least one substituent selected from halogen, C₁-C₄ alkyl, C₁-C₄ alkoxy, CN, OH, NO₂, C₁-C₃ alkyl substituted by one or more F atoms, C₁-C₃ alkoxy substituted by one or more F atoms, NR¹²R¹³, C≡R³⁰, CONR³¹R³², CHO, C₂-C₄ alkanoyl, S(O)ₚR³³ and OSO₂R³⁴;
R¹², R¹³, R⁴⁰ and R⁴¹ independently represent H, alkyl, formyl or alkylcarbonyl; or
the group —NR¹²_{R}¹³ or —NR⁴⁰R⁴¹ together represents a 5 to 7 membered azacyclic ring optionally incorporating one further heteroatom selected from O, S and NR³⁸;
R³⁰ and R⁴⁵ independently represent H, alkyl, Si(CH₃)₃ or phenyl;
R³³ and R³⁴ independently represent H or alkyl; said alkyl being optionally substituted by one or more F atoms;
R¹⁰ represents H or F;
R⁸ represents phenyl or a five- or six-membered heteroaromatic ring containing 1 to 3 heteroatoms independently selected from O, S and N; said ring being optionally substituted with at least one substituent selected from halogen, alkyl, cyano, alkoxy, nitro, methylcarbonyl, NR³⁵R³⁶, alkyl substituted by one or more F atoms or alkoxy substituted by one or more F atoms;
R³⁵, R³⁶, R⁴⁸ R⁴⁹ independently represent H or alkyl; said alkyl being optionally further substituted by one or more F atoms;
R⁹ represents hydrogen or alkyl optionally substituted with at least one substituent selected from fluoro, hydroxyl and alkoxy;
p is 0, 1 or 2;
s is 0, 1 or 2;
R³¹, R³², R³⁷, R³⁸, R⁴⁶ and R⁴⁷ each independently represent hydrogen or alkyl; and
→ indicates the preferred point of attachment of M² to the group L;
and each group M in formula (I) is selected independently from a group M¹ or M² provided that every compound of formula (I) contains at least one group M²;
L represents a linker group of formula (V):
-L^{I}-R¹⁵-L²-R¹⁶-L³-X-L³-R¹⁶-L²-R¹⁵-L¹- (V)
wherein:
each L¹, each L² and each L³ is independently selected from a direct bond, C(═O), O, NR¹⁷, CONR¹⁸ and NR¹⁹CO;
each R¹⁵ and each R¹⁶ is independently selected from C1 to 10 alkylene or C3 to 7 cycloalkylene; and
X is a direct bond, C(═O), NR²⁰R²¹, alkylene, cycloalkylene, aryl, aryl¹-aryl², aryl¹-O-aryl², heteroaryl, heteroaryl¹-heteroaryl², heteroaryl¹-O-heteroaryl² or is selected from the following divalent radicals:
—(O—R²²)ₙ—O—
—N(R²³)—R²²—N(R²⁴)(R²⁵)—R²²—N(R²³)—
—N(R²³)—R²²—N(R²⁷)—C(≡NR²⁶)—N(R²⁸)—R²²—N(R²³)—
—N(R²³)—R²²—N(R²³)—
—N(R²⁷)—C(≡NR²⁶)—N(R²⁸)— 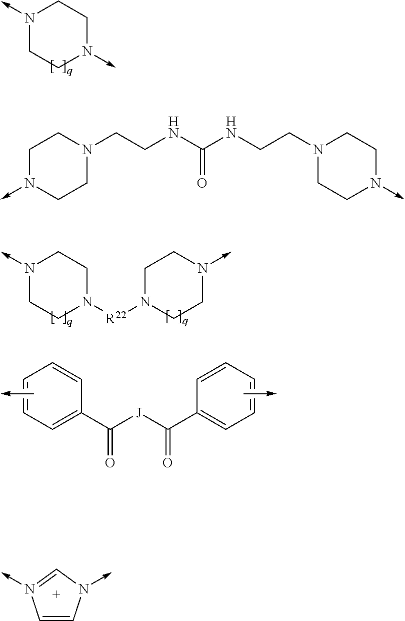
wherein
n is an integer 1 to 4;
each q independently represents an integer 1 or 2;
each R¹⁷, each R¹⁸ and each R¹⁹ are independently selected from H or alkyl;
R²⁰ and R²¹ are independently selected from H and alkyl; and when both represent alkyl, the N atom to which they are attached bears a positive charge; or
R²⁰ and R²¹ are joined together such that the group NR²⁰R²¹ together represents a quaternary 5- to 7-membered azacyclic ring which optionally incorporates one further heteroatom selected from O, N and S;
aryl¹ and aryl² represent the same or different aryl ring systems;
heteroaryl¹ and heteroaryl² represent the same or different heteroaryl ring systems;
each R²² is independently selected from C1 to 10 alkylene or C3 to 7 cycloalkylene;
each R²³, each R²⁶, each R²⁷ and each R²⁸ is independently selected from H or alkyl;
R²⁴ and R²⁵ are independently selected from H and alkyl; and when both represent alkyl, the N atom to which they are attached bears a positive charge; or
R²⁴ and R²⁵ are joined together such that the group NR²⁴R²⁵ together represents a quaternary 5- to 7-membered azacyclic ring which optionally incorporates one further heteroatom selected from O, N and S;
J is selected from the groups —N(R²³)—R²²—N(R²⁴)(R²⁵)—R²²—N(R²³)— or —N(R²³)—R²²—N(R²⁷)—C(≡NR²⁶)—(NR²⁸)—R²²—N(R²³)—;
or a pharmaceutically acceptable salt thereof.

2. A compound according to claim 1, wherein in formula (IIA) or (IIB) A represents a phenyl ring, D is O and each of Y¹ to Y⁵ is a carbon atom.

3. A compound according to claim 1 or claim 2, wherein R⁷ represents methyl; W represents S(O); Z represents a single bond; R¹⁴ represents phenyl optionally substituted by one or two substituents independently selected from cyano, F, Cl, Br, CF₃, NO₂ and —C≡CH; R¹⁰ represents H; R⁸ represents a phenyl group substituted with a trifluoromethyl substituent; and R⁴ represents hydrogen

4. A compound of formula (VI):
{(M)-(L⁴)]ₜ-G (VI)
wherein:
t represents an integer 3 to 20;
L⁴ represents a linker group of formula -L¹-R¹⁵-L²-R¹⁶-L³-wherein L¹, L², L³, R¹⁵ and R¹⁶ are as defined above;
G represents is N, aryl, aryl¹-aryl², aryl¹-O-aryl², heteroaryl, heteroaryl¹-heteroaryl², heteroaryl¹-O-heteroaryl², a dendrimer or is selected from the following multivalent radicals wherein R²² is as defined above and r is an integer 1 to 6: 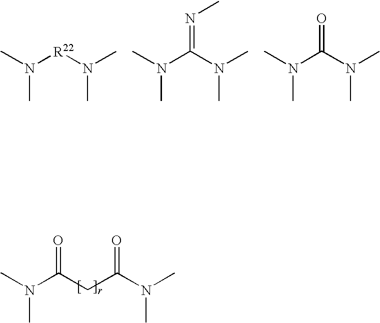
and M is as defined for formula (I) with the proviso that at least one M group represents M²;
or a pharmaceutically acceptable salt thereof.

5. A compound according to claim 4, wherein t represents an integer 3 to 5.

6. A compound of formula (I) as defined in claim 1 which is:
N,N′-[ethane-1,2-diylbis(oxyethane-2,1-diyl)]bis {5-[(4-cyanophenyl)sulfinyl]-6-methyl-2-oxo-1-[3-(trifluoromethyl)phenyl]-1,2 dihydropyridine-3-carboxamide};
N,N′-(2-hydroxypropane-1,3-diyl)bis(5-(1-(4-cyanophenyl)-1H-pyrazol-5-yl)-6-methyl-2-oxo-1-(3-(trifluoromethyl)phenyl)-1,2-dihydropyridine-3-carboxamide)
or a pharmaceutically acceptable salt of any one thereof.

7. A pharmaceutical composition comprising a compound of formula (I) or formula (VI) or a pharmaceutically acceptable salt thereof as claimed in any one of claims 1 to 6 in association with a pharmaceutically acceptable adjuvant, diluent or carrier.

8. A process for the preparation of a pharmaceutical composition as claimed in claim 7 which comprises mixing a compound of formula (I) or formula (VI) or a pharmaceutically acceptable salt thereof as claimed in any one of claims 1 to 6 with a pharmaceutically acceptable adjuvant, diluent or carrier.

9. A compound of formula (I) or formula (VI) or a pharmaceutically-acceptable salt thereof as claimed in any one of claims 1 to 6 for use in therapy.

10. Use of a compound of formula (I) or formula (VI) or a pharmaceutically acceptable salt thereof as claimed in any one of claims 1 to 6 in the manufacture of a medicament for the treatment of human diseases or conditions in which modulation of neutrophil elastase activity is beneficial.

11. Use of a compound of formula (I) or formula (VI) or a pharmaceutically acceptable salt thereof as claimed in any one of claims 1 to 6 in the manufacture of a medicament for use in treating adult respiratory distress syndrome (ARDS), cystic fibrosis, pulmonary emphysema, bronchitis, bronchiectasis, chronic obstructive pulmonary disease (COPD), pulmonary hypertension, asthma, rhinitis, ischemia-reperfusion injury, rheumatoid arthritis, osteoarthritis, cancer, atherosclerosis or gastric mucosa' injury.

12. A method of treating, or reducing the risk of, a disease or condition in which inhibition of neutrophil elastase activity is beneficial which comprises administering to a patient in need thereof a therapeutically effective amount of a compound of formula (I) or formula (VI) or a pharmaceutically acceptable salt thereof as claimed in any one of claims 1 to 6.

13. A method of treating, or reducing the risk of, an inflammatory disease or condition which comprises administering to a patient in need thereof a therapeutically effective amount of a compound of formula (I) or formula (VI) or a pharmaceutically acceptable salt thereof as claimed in any one of claims 1 to 6.

14. A method according to claim 12 or claim 13, wherein the disease or condition is adult respiratory distress syndrome (ARDS), cystic fibrosis, pulmonary emphysema, bronchitis, bronchiectasis, chronic obstructive pulmonary disease (COPD), pulmonary hypertension, asthma, rhinitis, ischemia-reperfusion injury, rheumatoid arthritis, osteoarthritis, cancer, atherosclerosis or gastric mucosal injury.
